(19) United States
(12) Patent Application Publication
(10) Pub. No.: US 2005/0187616 A1
(43) Pub. Date: Aug. 25, 2005

(54) Methods and apparatus for valve repair

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 10/990,342

(22) Filed: Nov. 15, 2004

Related U.S. Application Data
(60) Provisional application No. 60/520,197, filed on Nov. 13, 2003.

(51) Int. Cl. A61F 2/24

(52) U.S. Cl. 623/2.11; 227/175.1

(57) ABSTRACT

A valve delivery device is provided. The device comprises a heart valve prosthesis support having a proximal portion and a distal portion; a plurality of fasteners ejectably mounted on the support; a heart valve prosthesis being releasably coupled to said distal portion of said heart valve prosthesis support; and where the heart valve prosthesis and support are configured for delivery to the heart through an aortotomy formed in the patient's aorta. The device may include an anvil movable along a longitudinal axis of the device to engage tissue disposed between the anvil and the valve prosthesis.

The present application claims the benefit of priority from co-pending U.S. Provisional Patent Application Ser. No. 60/520,197 (Attorney Docket No. 40450-0002) filed on Nov. 13, 2003. This application is incorporated herein by reference for all purposes.

### BACKGROUND OF THE INVENTION

1. Technical Field

The invention relates to apparatus and methods for valve replacement and is especially useful in aortic valve repair procedures.

2. Background Art

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardiopulmonary bypass for an extended period of time.

Applicants believe that there remains a need for improved valvular repair apparatus and methods that use minimally invasive techniques and/or reduce time in surgery.

### SUMMARY OF THE INVENTION

The present invention involves valve repair apparatus and methods that overcome problems and disadvantages of the prior art. According to one aspect of the invention, minimally invasive valve removal apparatus is provided, which includes cutting elements configured for delivery to the valve through an aortotomy formed in the patient's aorta.

In one embodiment, heart valve leaflet removal apparatus of the present invention comprises a pair of cooperating cutting elements, a holder and members for manipulating the cutting elements. The cooperating cutting elements are adapted for cutting and removing leaflets from a heart valve, one of the cutting elements is rotatably coupled the other of the pair of cutting elements. The holder is coupled to one of the cutting elements and is adapted to receive the cut leaflets. The members are coupled to each of the cutting elements for manipulating the cutting elements. And the cutting elements and holder are configured for delivery to the valve leaflets through an aortotomy formed in a patient's aorta. In one variation, the pair of cooperating cutting elements and holder have a radial dimension and are radially collapsible.

According to one aspect of the invention, minimally invasive valve prosthesis delivery apparatus is provided, which includes a valve prosthesis support adapted for delivery to the valve through an aortotomy formed in the patient's aorta.

In one embodiment, heart valve prosthesis delivery apparatus of the present invention for placing heart valve prosthesis in a patient's heart comprises heart valve prosthesis support and heart valve prosthesis. The heart valve prosthesis support having a proximal portion and a distal portion and plurality of fasteners ejectably mounted therein. The heart valve prosthesis being releasably coupled to said distal portion of said heart valve prosthesis support. And the heart valve prosthesis and support being configured for delivery to the heart through an aortotomy formed in the patient's aorta.

In one embodiment, the present invention provides a valve delivery device. The device comprises a heart valve prosthesis support having a proximal portion and a distal portion; a plurality of fasteners ejectably mounted on the support; a heart valve prosthesis being releasably coupled to said distal portion of said heart valve prosthesis support; and where the heart valve prosthesis and support are configured for delivery to the heart through an aortotomy formed in the patient's aorta. By way of example and not limitation, the device may include a support device such as but not limited to an anvil or support device movable along a longitudinal axis of the device to engage tissue disposed between the anvil and the valve prosthesis.

In another embodiment, the present invention provides a valve delivery device for use with a stentless prosthesis. The device comprises a heart valve prosthesis support having a proximal portion and a distal portion; a plurality of fasteners ejectably mounted on the support; a stentless heart valve prosthesis being releasably coupled to said distal portion of the heart valve prosthesis support; and where the heart valve prosthesis and support being configured for delivery to the heart through an aortotomy formed in the patient's aorta. The device may include an anvil movable along a longitudinal axis of the device to engage tissue disposed between the anvil and the valve prosthesis.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail. A further understanding of the nature and advantages of the invention will become apparent by reference to the remaining portions of the specification and drawings.

A further understanding of the nature and advantages of the invention will become apparent by reference to the remaining portions of the specification and drawings.

### BRIEF DESCRIPTION OF THE DRAWINGS

FIG. 1 illustrates an aortic root pulled back to show the aortic valve leaflets to be removed in an aortic valve replacement procedure of the present invention;

FIG. 2A is perspective view of minimally invasive valve cutting apparatus suitable for removing the valve leaflets from an aortic valve in accordance with the present invention and shown in a collapsed state;

FIG. 2B is a perspective view of the apparatus of FIG. 2A shown in an expanded state and illustrated for exemplary purposes positioned in an aortic valve;

FIG. 2C is a perspective view of the apparatus of FIG. 2B illustrating the cutting members of the apparatus engaged after cutting the aortic valve leaflets from the aortic valve;

FIG. 3A is a perspective view of another minimally invasive valve cutting apparatus in accordance with the present invention;

FIGS. 3B, 3C, and 3D are diagrammatic partial sectional views of the apparatus of FIG. 3A where FIG. 3B shows the pair of cooperating cutting elements of the apparatus above the valve leaflets, FIG. 3C shows one of the cooperating cutting elements positioned below the valve leaflets, and FIG. 3D shows the upper cooperating cutting element rotated and the valve leaflets separated form the original valve;

FIG. 4A is a perspective view of valve prosthesis and clip delivery apparatus in accordance with the invention shown supporting valve prosthesis and being in a collapsed state for minimally invasive delivery of the valve prosthesis (e.g., through an aortotomy);

FIG. 4B is another perspective view of the delivery apparatus of FIG. 4A with the support arm slide retracted to place the arms in an expanded state;

FIG. 4C is another perspective view of the delivery apparatus of FIG. 4A with the clip ejection actuator moved distally to eject the fasteners, which fasten the valve prosthesis to the surgical site;

FIG. 4D is another perspective view of the delivery apparatus of FIG. 4A illustrating removal of the delivery apparatus after the clips have been released;

FIGS. 5A-5D are partial sectional views of the distal end of the delivery apparatus of FIG. 4A and the valve prosthesis seated on an aortic valve diagrammatically illustrating clip delivery where FIG. 5A shows the ends of the support arms penetrated through the sides of the replacement valve, FIG. 5B shows the ejection of the clips into the aortic root wall, FIG. 5C illustrates withdrawal of the ends of the support arms and the clips fully released and securing the valve prosthesis to the aortic valve annulus, and FIG. 5D illustrates complete removal of the prosthesis and clip delivery apparatus;

FIG. 5E is a detailed view illustrating a pusher member of the valve prosthesis and clip delivery apparatus ejecting a clip;

FIG. 5F illustrates the clip of FIG. 5E discharges from the delivery apparatus support arm and in place where it secures a portion of the valve prosthesis to the aortic annulus;

FIG. 6 illustrates how the valve prosthesis attachment would appear if the aortic root were cut and pulled back after implantation;

FIG. 7 illustrates placement of an expandable balloon within the valve prosthesis after the valve prosthesis is secured to the aortic annulus with the balloon expanded and compressing the outer wall surfaces of prosthesis having bio-glue applied thereto against the aortic inner wall;

FIG. 8 is a perspective view of the delivery apparatus of FIG. 4A supporting a mechanical valve;

FIG. 9A is a side view of the mechanical valve of FIG. 8 in an open state;

FIG. 9B is a side view of the mechanical valve of FIG. 8 in a closed state;

FIG. 10 is a perspective view of the mechanical valve secured to the aortic annulus after delivery with the delivery apparatus of FIG. 9; and

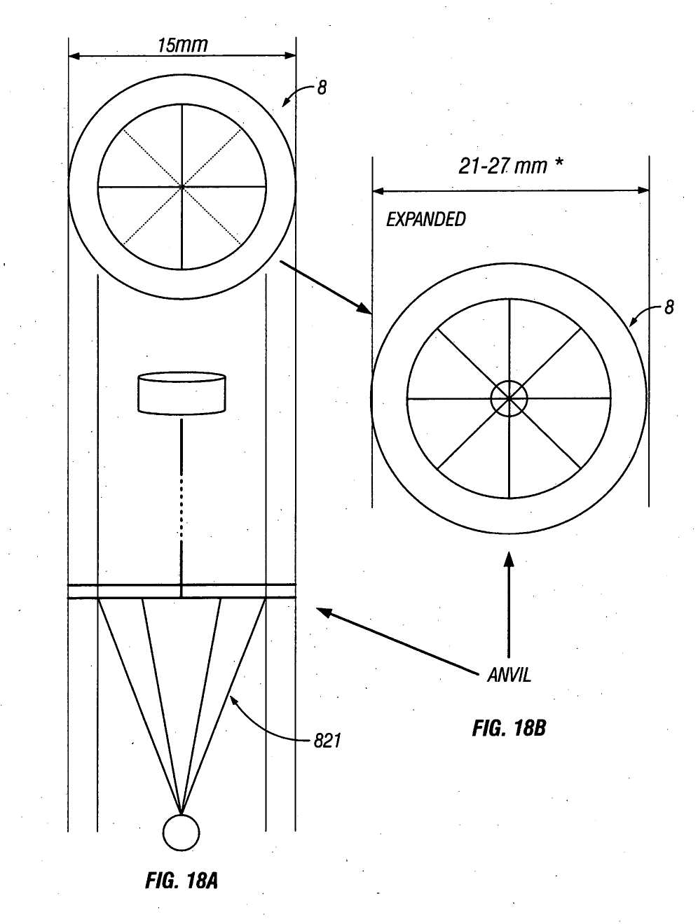

FIG. 11 is a top plan view the fastener clip depicted in various of the foregoing Figures shown in a relaxed or free state.

FIG. 12 shows a prosthesis delivery device for use with a support device.

FIGS. 13 and 14 show one embodiment of the support device.

FIG. 15 shows the support device of FIG. 13 in the heart.

FIG. 16 shows the support device used to engage tissue between itself and a prosthetic.

FIG. 17 shows fasteners coupling a prosthetic against a target tissue.

FIGS. 18A-B show one embodiment of an expandable support device.

FIGS. 19-20 show various views of another embodiment of the present invention.

FIGS. 21-23 show side cross-sectional view of various prosthesis delivery devices.

FIG. 24 is another cross-sectional view of one device according to the present invention.

FIG. 25 is another cross-sectional view of one device according to the present invention.

FIGS. 26A-26B are still further cross-sectional views of a device according to the present invention.

FIG. 27 shows one position of a valve prosthesis against an annulus and a comparison of larger valves that can be used with the present attachment technique.

FIGS. 28-29 show various positions for aligning a valve prosthetic according to the present invention.

FIGS. 30-31 show the use of alignment sutures.

FIG. 32 is cross-sectional view showing delivery of one fastener.

FIGS. 33A-33C show various views of one fastener according to the present invention.

FIGS. 34-37 show the delivery of a fastener device according to the present invention.

FIGS. 38-42 show the use of another fastener embodiment according to the present invention.

FIGS. 43 and 44 show a ring with a plurality of fasteners.

FIGS. 45 and 46 show various views of another prosthesis delivery device according to the present invention.

FIG. 47 shows one embodiment of a support device according to the present invention.

FIG. 48 shows one embodiment of a fastener housing according to the present invention.

FIGS. 49-50 show various views of the device of FIG. 46.

FIG. 51 shows a cross-sectional view of yet another embodiment of a delivery device according to the present invention.

FIG. 52 shows a valve prosthesis without a sewing ring.

FIG. 53 shows an enlarged cross-sectional view of the device of FIG. 51.

FIG. 54 shows a portion of one embodiment of the hollow sharpened member.

FIG. 55 shows a cross-section of one embodiment of a fastener housing.

FIG. 56 and 57 show enlarged cross-sectional views of a fastener being delivered to secure a prosthesis.

FIGS. 58 and 59 show other embodiments of devices for ejecting the fasteners.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for capturing debris, this means that the capture feature may or may not be present, and, thus, the description includes structures wherein a device possesses the capture feature and structures wherein the capture feature is not present.

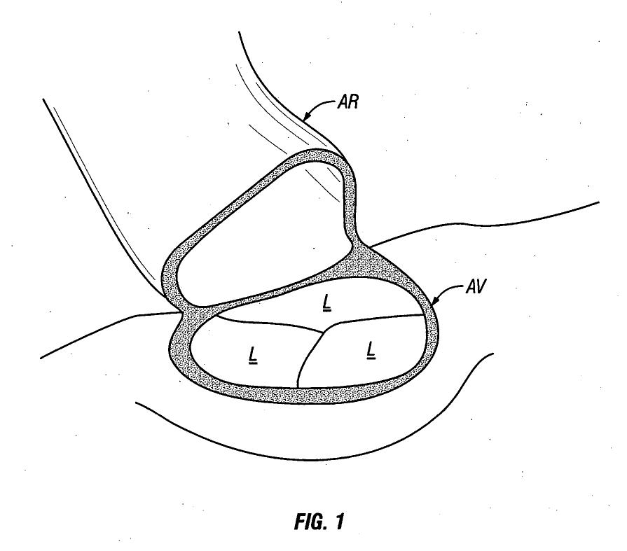

Referring to FIG. 1, an aortic root (AR) is shown pulled back to show the right, left, and posterior leaflets (L) of an aortic valve (AV) to be removed in a minimally invasive valve replacement procedure of the present invention where valve leaflet removal and valve prosthesis delivery apparatus can be delivered to the aortic root via an aortotomy.

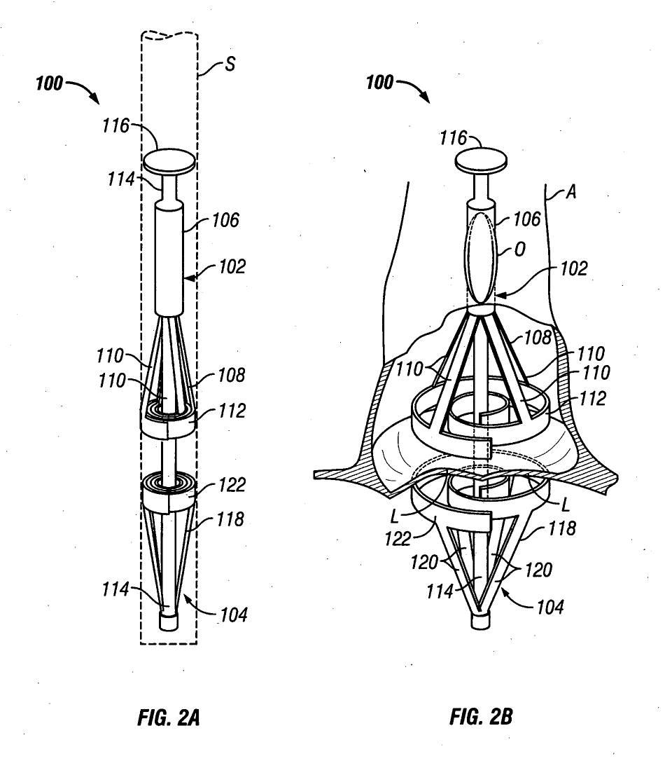

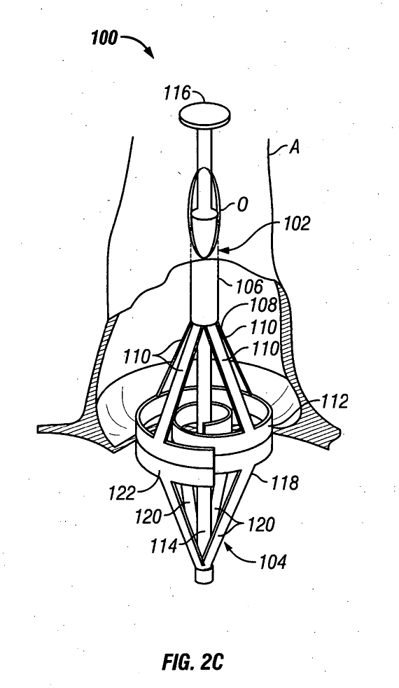

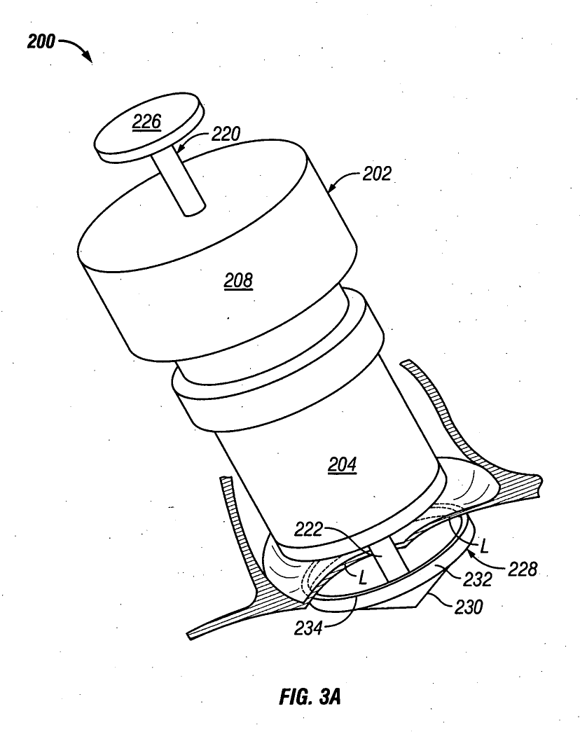

Referring to FIGS. 2A-C, one embodiment of minimally invasive valve cutting or removal apparatus is shown and generally designated with reference numeral 100. Apparatus 100 includes a first body member 102 and a second body member 104. First body member 102 includes a tubular member 106 and an umbrella having umbrella arms 110 and a cutting element 112, which is in the form of a spiral. Cutting element 112 can be formed from flat metal wire, such as flat stainless steel wire or ribbon or any other materials suitable cutting. Umbrella arms 110 each have one end secured to or integrally formed with tubular member 106 and one end secured to or integrally formed with cutting element 112.

Second body member 104 includes and elongated member 114, which can include a knob 116 at one end thereof. Second body member 104 also includes an umbrella 118, which is similar to umbrella 108. Umbrella 118 includes umbrella arms 120 and umbrella cutting element 122, which also is in the form of a spiral. Cutting element 122 can be formed from flat metal wire, such as flat stainless steel wire or ribbon or any other material suitable for cutting. Umbrella arms 120 each have one end secured to or integrally formed with elongated member 114 and one end secured to or integrally formed with cutting element 122.

As shown in FIG. 2A, the first and second umbrellas 108 and 118 are radially compressible or collapsible. A tube or sheath such as shown in dashed lines and indicated with reference character “S” in FIG. 2A can be placed around apparatus 100 to hold it in a collapsed state. With the sheath in place so that the umbrellas are in the radially compressed or collapsed state, where the umbrellas have a radial dimension less that of their uncompressed or uncollapsed state as shown in FIGS. 2A and 2B, sheath S and valve removal apparatus 100 are introduced through an opening 0 or aortotomy formed in the aorta (A) of a patient. When the second umbrella is positioned below the aortic leaflets (L) and the first umbrella is positioned above the aortic leaflets (L), the umbrellas are allowed to expand to their memory or relaxed state shown in FIG. 2B by retracting the sheath. If the umbrellas are not aligned as shown in FIG. 2A, members 106 and 114 can be manipulated to adjust the umbrella positions. Other mechanisms for holding elements 112 and 122 or the umbrellas radially compressed can be used. For example, a wire can be wrapped around elements 112 and 122 and pulled away from the apparatus when the umbrellas are in place and ready to deploy.

Referring to FIG. 2C, tubular member 106 and elongated member 114 are then moved in opposite directions to compress the leaflets between the opposed cutting edges of cutting elements 112 and 122, which edges can be sharpened to enhance cutting. Tubular member 106 and/or elongated member 114 also can be rotated to complete the cut if necessary. The cut leaflets can fall into second umbrella 118, which forms a holder for the leaflets if they do not remain between the cutting edges during removal of the apparatus.

Before removing the apparatus 100, it again is radially compressed. This can be done by sliding sheath S through over apparatus 100. If the second umbrella does not close with the first umbrella, the surgeon retract the apparatus so that the second umbrella is in the vicinity of the aortotomy and manipulate spiral cutting element 122 to reduce the diameter of the second umbrella. In this manner, apparatus 100, together with the cut leaflets are removed from the site through the aortotomy.

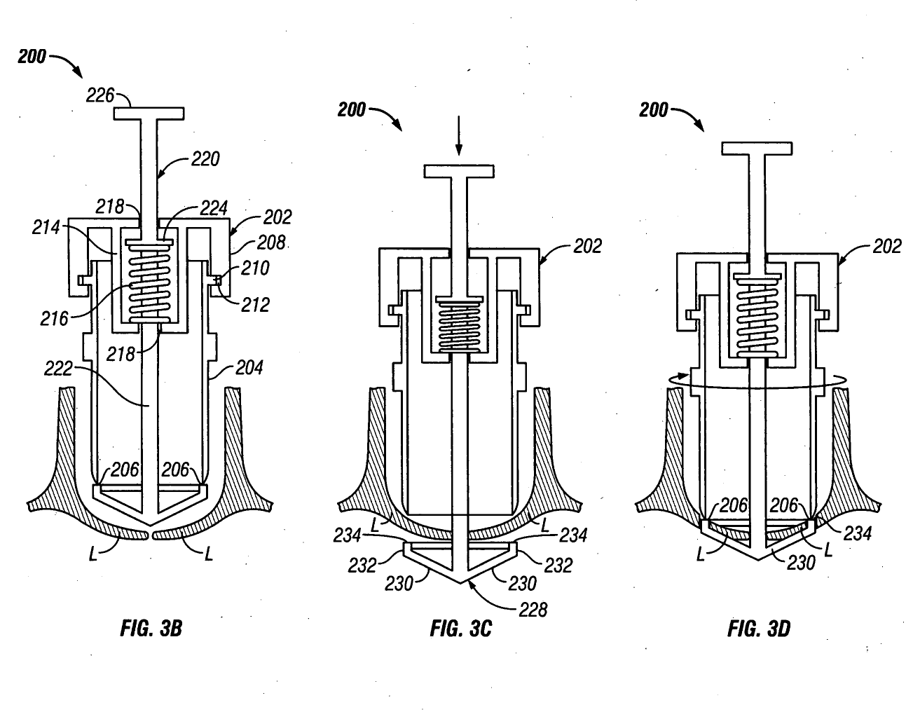

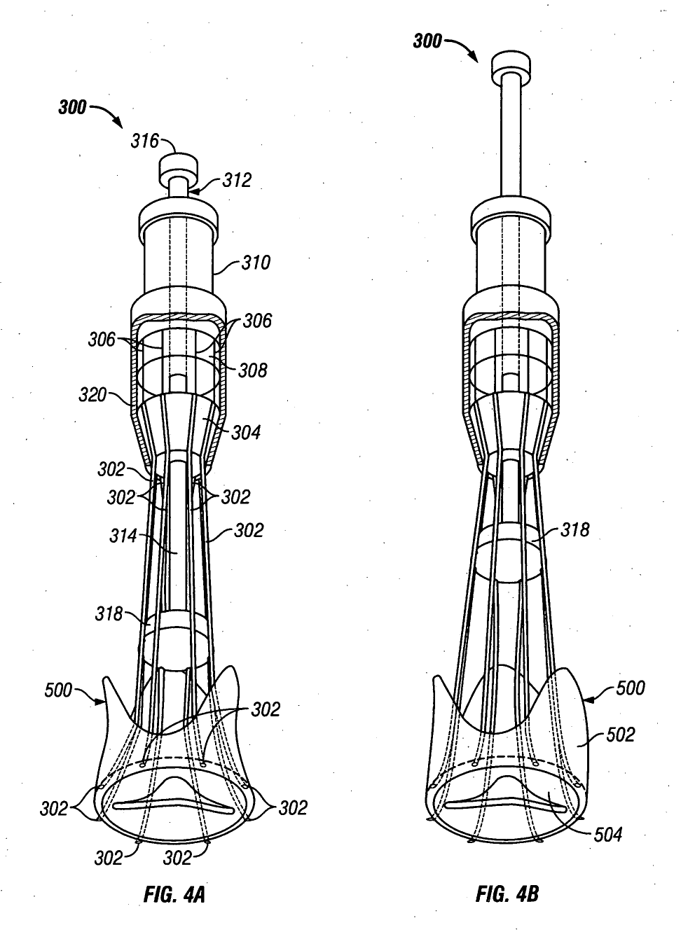

Referring to FIGS. 3A-D, another minimally invasive valve cutting or removal apparatus in shown accordance with the present invention and generally designated with reference numeral 200. Valve removal apparatus 200 generally includes a housing 202 and plunger 220 slidably mounted therein.

Housing 202 includes a first tubular portion or member 204, which has an annular cutting edge or element 206 at the distal end thereof, and a second portion or member 208 coupled thereto or integrally formed with first portion or member 204. First and second portions or members 204 and 206 can be rotatably coupled to one another through an annular tongue 210 and groove 212 arrangement as shown in FIGS. 3B-D. However, other coupling arrangements can be used and members 204 and 206 can be fixedly secured to one another. Second member or portion 208 includes a chamber 214 that houses and supports spring 216 and includes vertically aligned holes 218 through which plunger 220 is slidably mounted.

Plunger 220 includes an elongated member or rod 222 having an enlarged disc shaped portion 224 for interfacing with spring 216, a handle or knob 226 and a cutting and leaflet holding member 228 that cooperates with cutting edge 206. In the illustrative embodiment, cutting member 228 includes conical section 230 and cylindrical section 232, which forms annular cutting block or surface 234. Annular surface or element 234 cooperates with annular cutting edge or element 206 to cut the valve leaflets.

The distal portion of leaflet removal apparatus 200, which is adapted for passage through an aortotomy, is passed through such an aortotomy and positioned above the aortic valve leaflets a shown in FIG. 3B. Referring to FIG. 3C, the plunger is pressed or translated to position plunger cutting block 234 below the aortic leaflets. Compression spring 216 is allowed to return toward its relaxed state to drive the plunger proximally and squeeze the leaflets between surface 234 and cutting edge 206. In this position, housing portion 204 is rotated, as indicated with the arrow in FIG. 3D, to cut the leaflets. The cut leaflets fall into conical section or holder 230, which holds the cut leaflets as apparatus 200 is removed from the aortotomy.

According to another aspect of the invention, valve prosthesis delivery apparatus is provided to rapidly deliver the valve prosthesis to the surgical site and to secure the prosthesis at the desired location.

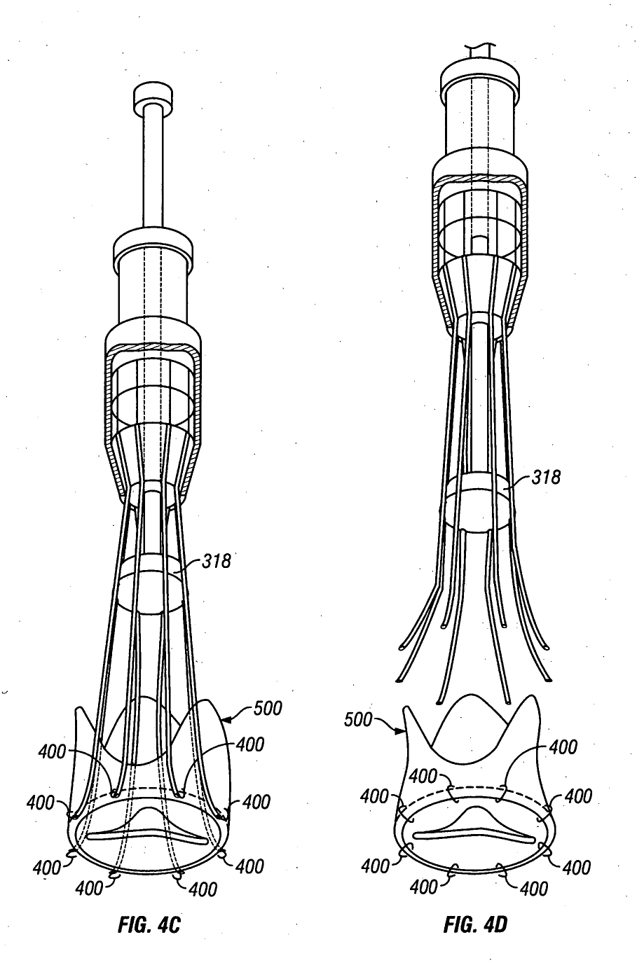

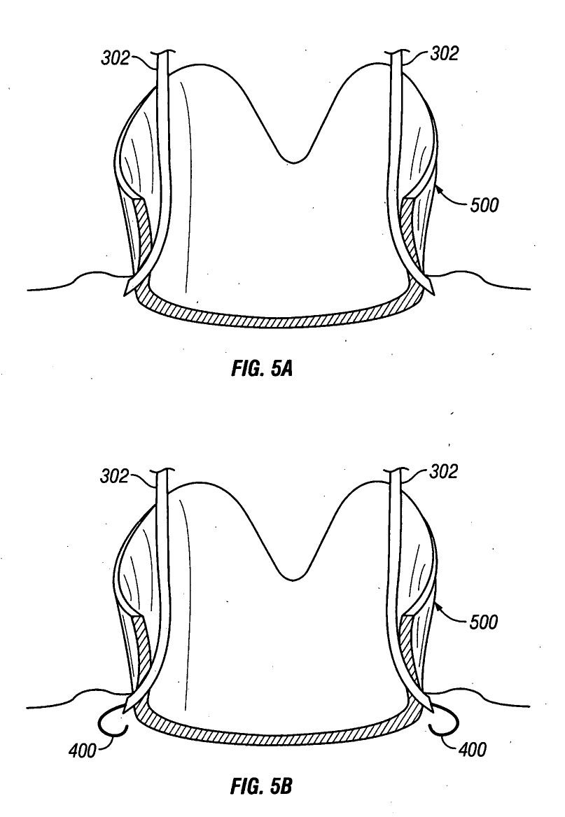

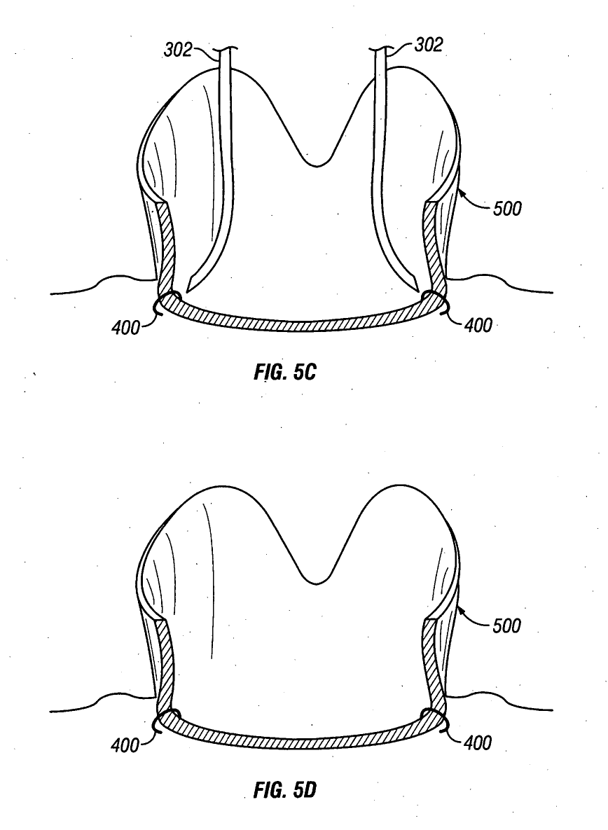

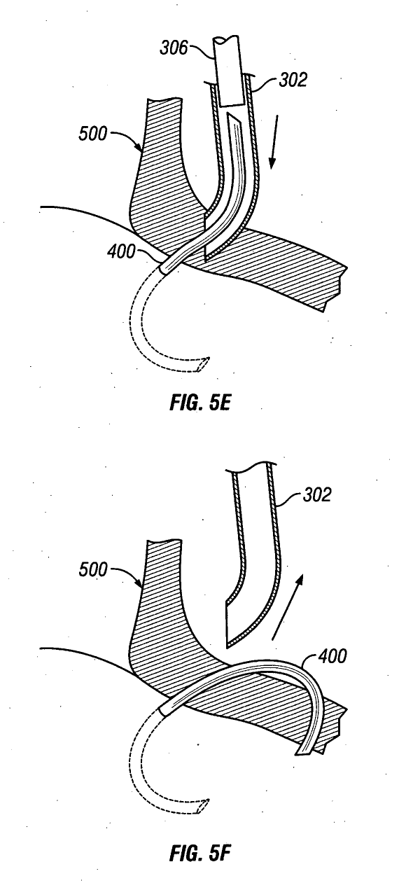

Referring to FIGS. 4A-C, an exemplary embodiment of a valve prosthesis delivery mechanism, which is generally designated with reference numeral 300, is shown. Valve prosthesis delivery apparatus 300 generally includes a support for supporting the prosthesis and a plurality of fastener ejectably mounted in the support.

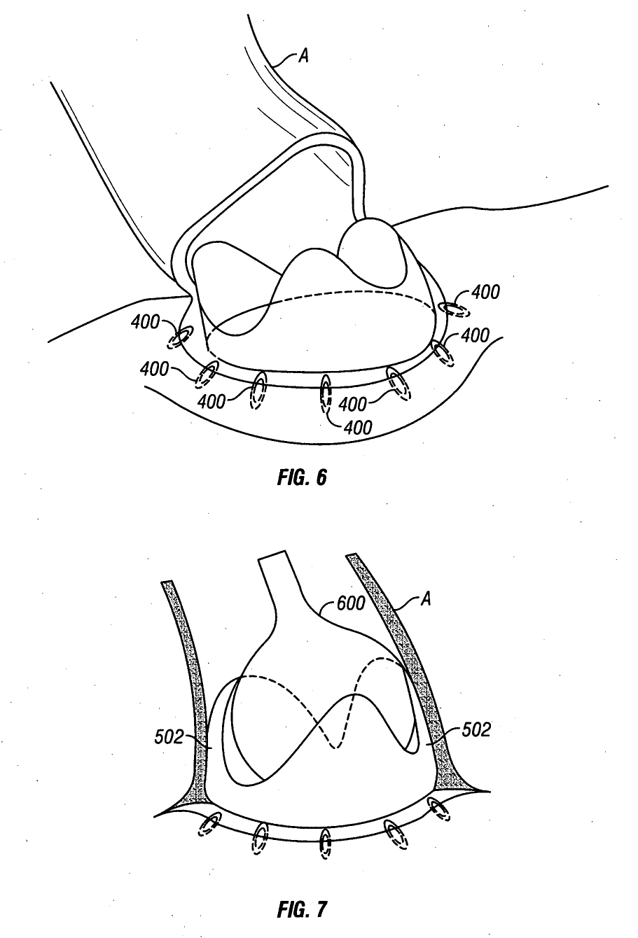

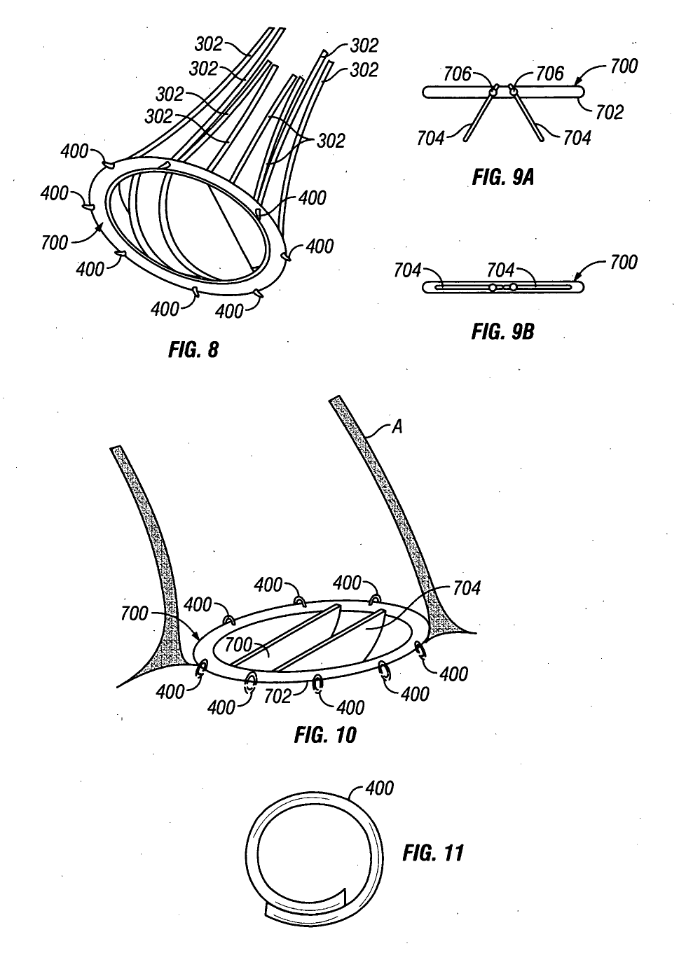

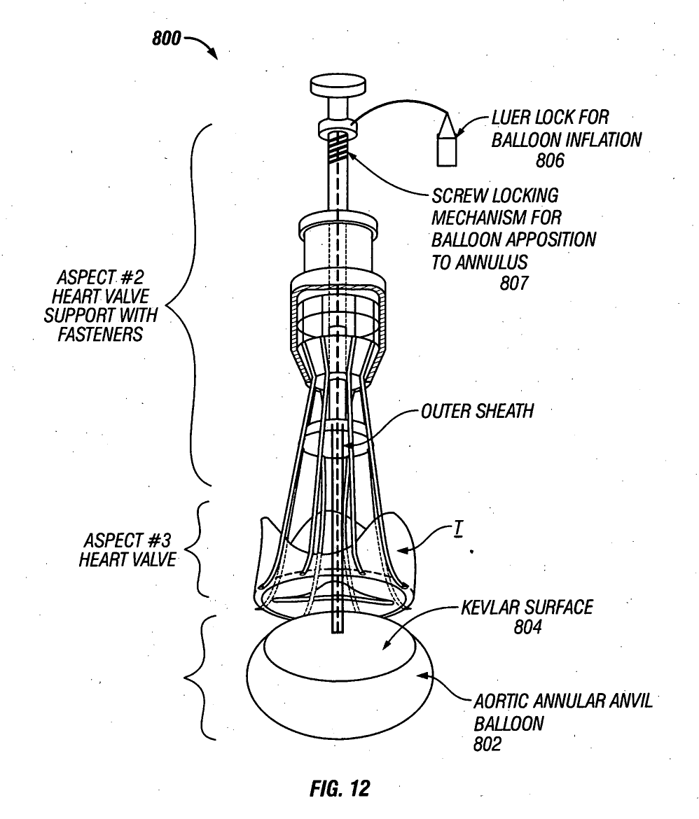

Referring to FIG. 4A, valve prosthesis mechanism 300 includes a prosthesis support comprising a plurality of tubes 302, each having a free distal end and a proximal portion fixedly secured to member 304, which in the illustrative embodiment, is frustoconical. A wire or pusher 306 is slidably mounted in each support tube 302 and includes a proximal portion that extends therefrom and is fixedly secured to plug 308, which can have the disc shape shown in the drawings. Grooves can be formed in member 304 and plug 308 for receiving support tubes 302 and wires 306, which can be formed form metal such as stainless steel, which has desirable stiffness. However, other suitable materials including nitinol can be used. Tubes 302 and wires 306 can be secured in the grooves by compressing sizing the grooves to be slightly smaller than the tubes and/or wires and/or by gluing. Plug 308 can be secured to cylindrical member 310 or integrally formed therein and form a portion thereof. Accordingly, when cylindrical member 310 is moved distally, wires 306 move distally to eject fastener clips 400 from support tubes 302 as shown in FIGS. 5E and 5F.

Valve prosthesis delivery apparatus 300 also can include apparatus or a mechanism for expanding support tubes 302 radially outward. In the illustrative embodiment, apparatus 300 includes a plunger 312, which includes elongated member 314. Elongated member 314 has a knob 316 at its proximal end and a slide member 318 at its distal end. Slide member 318 has a plurality of grooves formed therein in which support tubes 302 are slidably mounted. Slide member 318 is sized and/or configured so that when plunger 312 is moved proximally with slide member 318, slide member 318 urges support tubes radially outward. Plug 308 can be slidably mounted in a tubular housing 320, which can be secured to frustoconical member 304 as shown in the drawings. Housing 320 also is configured to slidably receive cylinder 310.

In use, valve prosthesis such as valve prosthesis 500 is secured to valve prosthesis delivery apparatus 300. Valve prosthesis 500 is shown as a conventional stentless tissue valve, which can be harvested from a suitable animal heart such as a porcine heart and prepared according to known methods. Valve prosthesis 500 includes a root portion 502 and a valve leaflet portion 504, which is shown in the drawings in an open position. In a closed configuration, the valve leaflet edges coapt to seal the valve and prevent regurgitation.

When securing valve prosthesis 500 to delivery apparatus 300, sliding member 318 is moved distally to allow the support tubes to return to their radially inward biased position as shown in FIG. 4A. Valve prosthesis 500 is then mounted on apparatus 300 so that a sharp pointed distal end of each support tube 302 extends through,the lower wall portion of tissue valve prosthesis 500.

Referring to FIGS. 4A-D, FIG. 4A, sliding member 318 can be advanced to allow the support arms to move radially inward to a collapsed state as a result of the biasing effect of frustoconically shaped plunger member 304. This position is used to introduce the apparatus through an aortotomy to the surgical site. FIG. 4B shows sliding member 318 retracted to place the arms in a radially expanded state. FIG. 4C shows cylinder 310 moved distally to eject the fastener clips 400, which are self-closing clips and fasten the valve prosthesis to the heart. FIG. 4D illustrates removal of the delivery apparatus after the clips have been released.

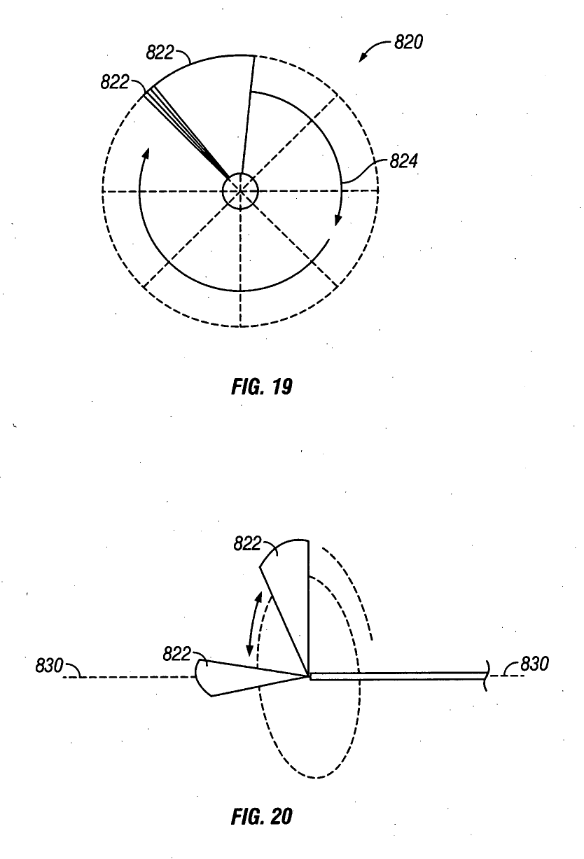

Self-closing clips 400 can comprise wire made from shape memory alloy or elastic material or wire so that it tends to return to its memory shape after being released from the clip delivery apparatus. As is well known in the art, shape memory material has thermal or stress relieved properties that enable it to return to a memory shape. For example, when stress is applied to shape memory alloy material causing at least a portion of the material to be in its martensitic form, it will retain its new shape until the stress is relieved as described in U.S. Pat. No. 6,514,265 to Ho et al. and which is hereby incorporated herein by reference. Then it returns to its original, memory shape. Accordingly, at least a portion of the shape memory alloy of clip 400 is converted from its austenitic phase to its martensitic phase when the wire is in its deformed, open configuration inside the curved distal end portion of a respective tube 302 (see e.g., FIG. 5E). When the stress is removed and clip 400 unrestrained, the material undergoes a martensitic to austenitic conversion and springs back to its undeformed configuration (FIG. 11).

One suitable shape memory material for the clip 400 is a nickel titanium (nitinol) alloy, which exhibits such pseudoelastic (superelastic) behavior.

The clip can be made by wrapping a nitinol wire having a diameter in the range of about 0.003 to 0.015 inch, and preferably 0.010 inch, and wrapping it around a mandrel having a diameter in the range of about 0.020 to 0.150, and preferably 0.080 inch. The heat treatment of the nitinol wire to permanently set its shape as shown in FIG. 11 can be achieved by heat-treating the wire and mandrel in either a convection oven or bath at a temperature range of 400 to 650° C., preferably 520° C., for a duration of 1 to 45 minutes, and preferably 15 minutes.

The following example is set forth with reference to FIGS. 5A-5E, 6, and 7 to further illustrate operation of valve prosthesis delivery apparatus 300 in replacing a malfunctioning aortic valve. It should be understood, however, that this example is not intended to limit its scope of the invention.

A patient is placed on cardiopulmonary bypass and prepared for open chest/open heart surgery, which typically requires a stemotomy. The surgeon removes the aortic leaflets using valve removal apparatus 100 or 200 as described above. Once the valve has been excised and removed with the valve removal apparatus, the surgeon then places a conventional aortic gazer through the aortotomy to determine the size of the aortic valve replacement (e.g., valve prosthesis 500) as is known in the art.

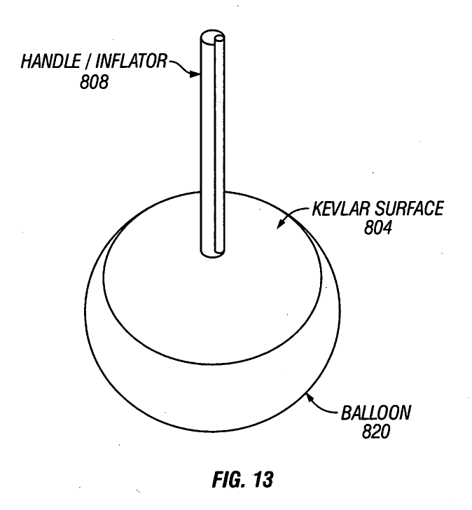

While in the generally collapsed state shown in FIG. 4A, valve prosthesis apparatus 300 is introduced through the aortotomy and the valve aligned with its natural location just below the two coronary arteries as is known in valve surgery. The sliding member 318 is retracted to have the piercing ends of support tubes 302 penetrate into the aortic root tissue as shown in FIG. 5A where the aorta is not shown for purposes of simplification. With valve prosthesis 500 seated and the sharp distal ends of the support arms 302 penetrated through the sides of the replacement valve 500 and slightly pushed further into adjacent the wall tissue, clips 400 are ejected into the adjacent wall tissue as shown in FIG. 5B. Specifically, cylinder 310 is moved distally so that pushers or wires 306 eject all of the clips 400 simultaneously (see FIGS. 4C and 5E). This one shot clip delivery can significantly reduce the time required to implant valve prosthesis as compared to other known techniques. After the clips are fully released and have tended to move toward their memory shape to secure valve prosthesis 500 in place as diagrammatically shown in FIG. 5C and more particularly in FIG. 5F, valve prosthesis delivery apparatus 300 is removed leaving the replacement valve secured at the desired site (FIG. 5D). FIG. 6 illustrates how the valve prosthesis attachment would appear if the aortic root were cut and pulled back after implantation.

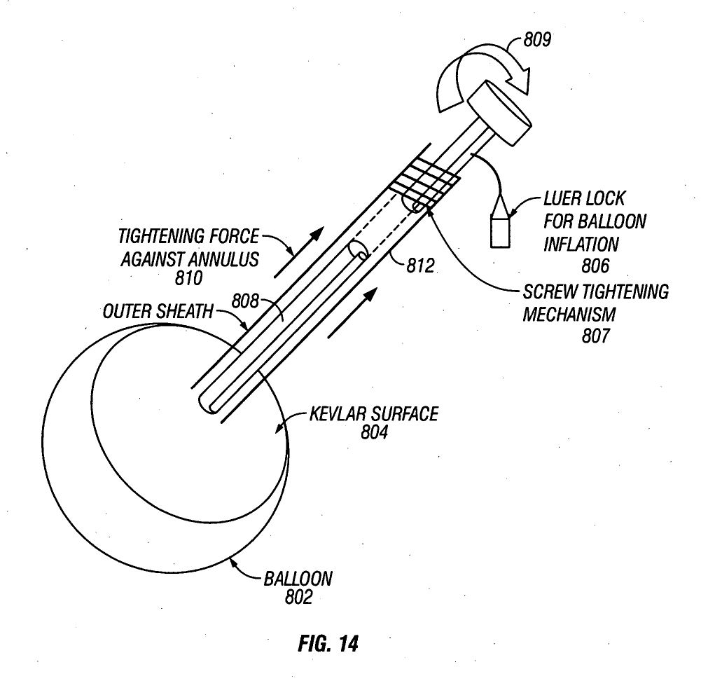

Referring to FIG. 7, a conventional aortic balloon catheter including a balloon, such as balloon 600, is used to urging the outer surface of the root of the valve prosthesis against the inner wall of the aorta. Before introducing the valve prosthesis through the aortotomy, the outer surface of the root of the valve prosthesis is coated with bio glue. Accordingly, as the balloon is expanded, it compresses the outer wall surfaces of prosthesis aortic root and the bio-glue applied thereto against the aortic inner wall and can hold it there while the glue sets. After the glue sets, the balloon is deflated and removed from the aortotomy and the aortotomy closed by conventional means.

Although the foregoing method has been described in connection with open chest surgery, the leaflet removal apparatus and prosthesis delivery apparatus described herein can be used with minimally invasive approaches that typically require a thotacotomy between adjacent ribs. Further, although the minimally invasive valve prosthesis replacement procedure has been described with reference to one prosthetic tissue valve, it should be understood that variations of such prosthesis or other valve prosthesis types can be used.

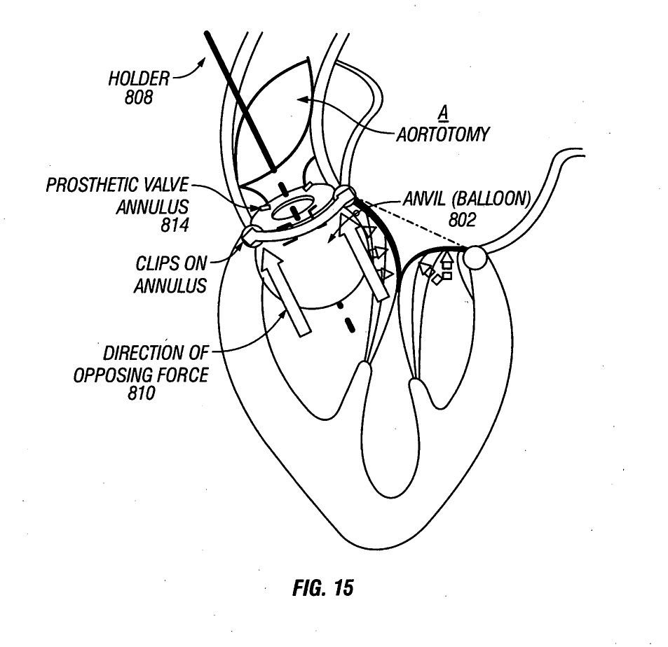

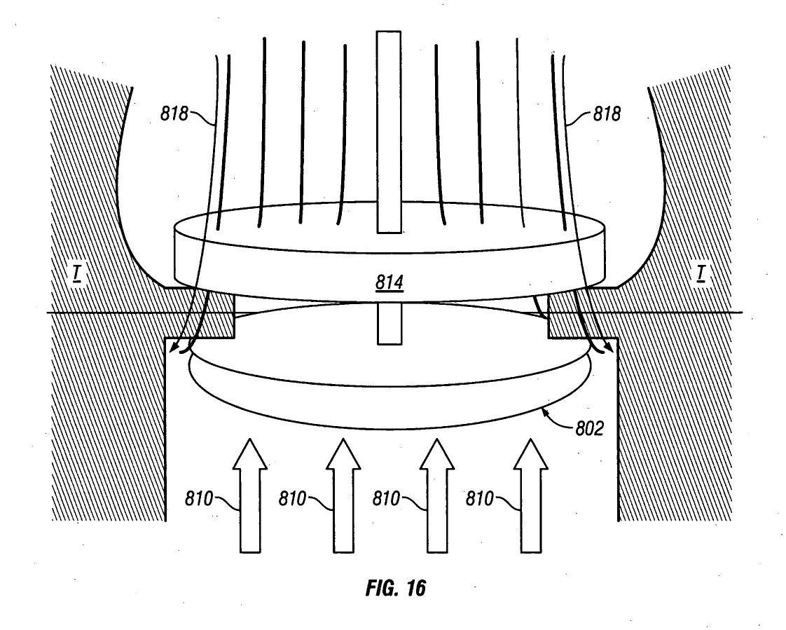

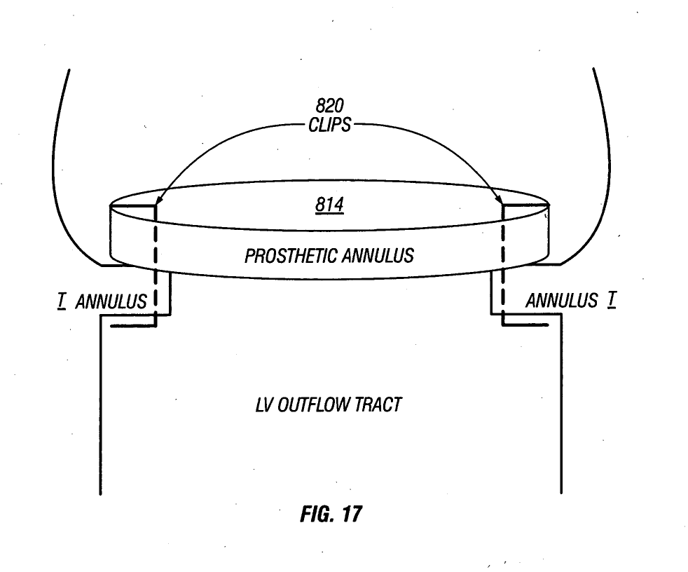

Referring to FIG. 8, valve prosthesis delivery apparatus 300 is shown in combination with a conventional mechanical hart valve prosthesis generally designated with reference numeral 700. Mechanical heart valve prosthesis 700 comprises an annular ring or housing 702, which can be metal or carbon material, to which two valve leaflets 704 are pivotally mounted. Each leaflet is pivotally mounted to ring 702 with two pivots 706 (two of the four pivots being hidden from view in FIG. 9A). A portion of each leaflet extends beyond its respective pivot as shown In FIG. 9A so that the leaflets can fully close the valve opening that ring 702 forms. Although a particular mechanical heart valve prosthesis is shown, it should be understood that any suitable mechanical heart valve prosthesis (or other valve prosthesis) can be used without departing from the scope of the invention. For example, a mechanical valve having a ball can be used.

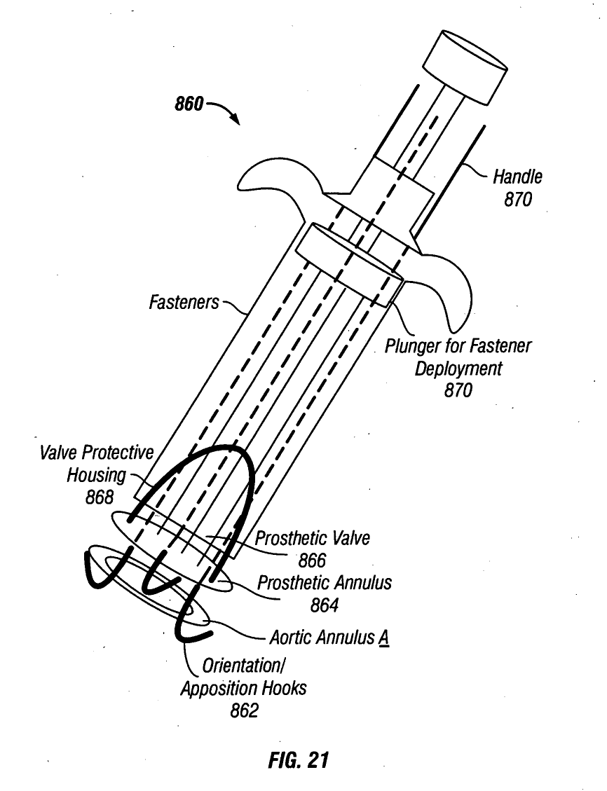

Referring now to FIG. 12, a still further embodiment of the present invention is shown. In this embodiment, an apparatus 800 is shown with an aortic anvil balloon 802. This balloon 802 is used to engage and/or grasp tissue T while clips and fasteners are being advanced by the apparatus 800. The balloon 802 may be, but is not necessarily, integrated with the apparatus 800. In this particular embodiment, the balloon 802 is inflatable to secure tissue between the balloon and the apparatus, thus facilitating delivery of sutures and/or clips through the tissue. Use of the balloon 802 may improve consistency and repeatability of suture and/or clip delivery since the targeted tissue may be grasped prior to engagement by the suture and/or clip. At least a portion 804 of the balloon 802 may be covered with a material, such as but not limited to Kevlar, DARON, Dacron, a firm rubber substance, GORTEX, any combination of the above, or similar substances to prevent clips or penetrating members from bursting the balloon during delivery into the tissue. In this embodiment, a Kevlar shield 804 may be used with the balloon 802. As seen in FIG. 12, a luer lock 806 may provided to enable inflation and/or deflation of balloon 802. It should be understood that during delivery, the balloon 802 may be in an uninflated condition to fascilitate entry and positioning of the balloon. In this embodiment, a screw locking mechanism 807 may used for balloon apposition to the annulus A or target tissue T. This may occur during, before, or after inflation of balloon 802.

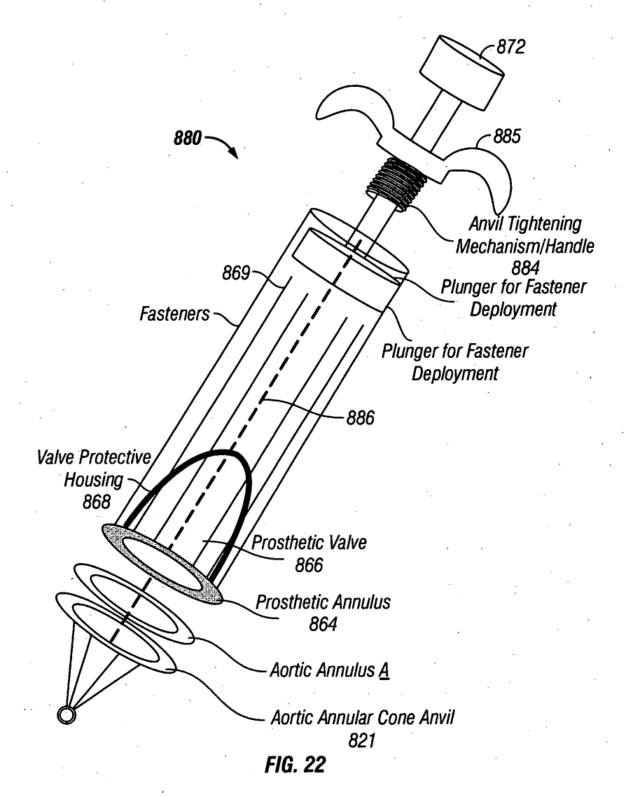

FIG. 13 provides an isolated view of just the balloon 802 in an inflated condition. As seen in FIG. 13, needle or fastener proof surface 804 may be provided on the balloon 802. A handle and/or balloon inflator 808 is also provided to enable positioning and inflation of the balloon.

FIG. 14 shows how the apparatus 800 functions with a balloon 802. In this embodiment, after inflation of balloon 802, tightening force may be provided through rotation of the screw tightening mechanism 807. As indicated, the screw mechanism 807 may be rotated as indicated by arrow 809. Tightening will cause the balloon 802 and its surface 804 to be retracted in the direction indicated by arrows 821. It should be understood that a variety of other mechanisms besides the screw such as but not limited to a ratchet mechanism or other retractor may be used to retracted the inflated balloon 802 in the direction 810. As seen in FIG. 14, an outer sheath 812 may be included for packaging purposes and to contain the various elements such as the tightening mechanism 807 and handle/inflation device 808. In this embodiment, the outer sheath provides counter traction between balloon and native annulus.

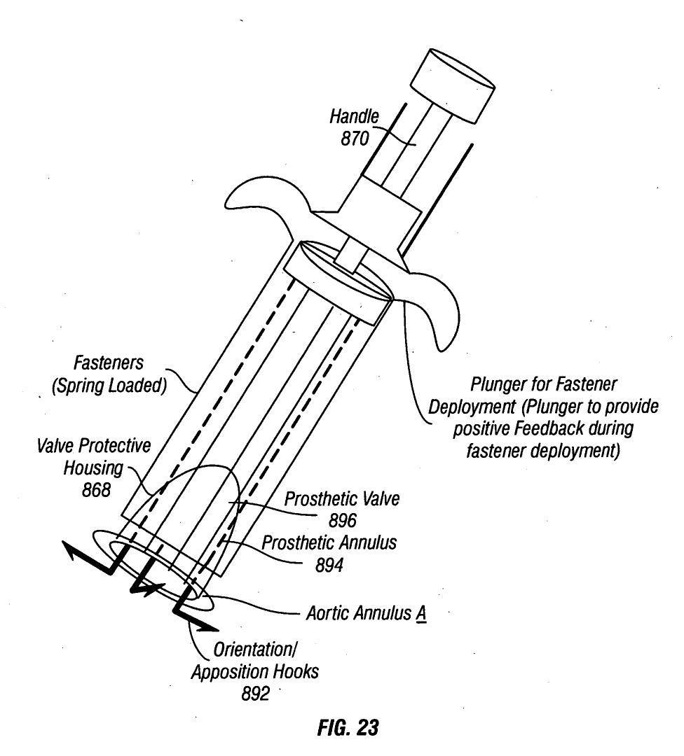

FIG. 15 shows the balloon 802 in use for an aortic valve procedure. As seen in FIG. 15, an aortotomy A is formed to provide access to the aortic valve area. The holder 808 is used to position the balloon 802. The inflated balloon 802 is drawn in the direction 810. This traps tissue T between the balloon 802 and the prosthetic valve annulus 814. In this particular embodiment, clips 816 are then delivered to secure the prosthetic valve annulus 814 to the tissue T.

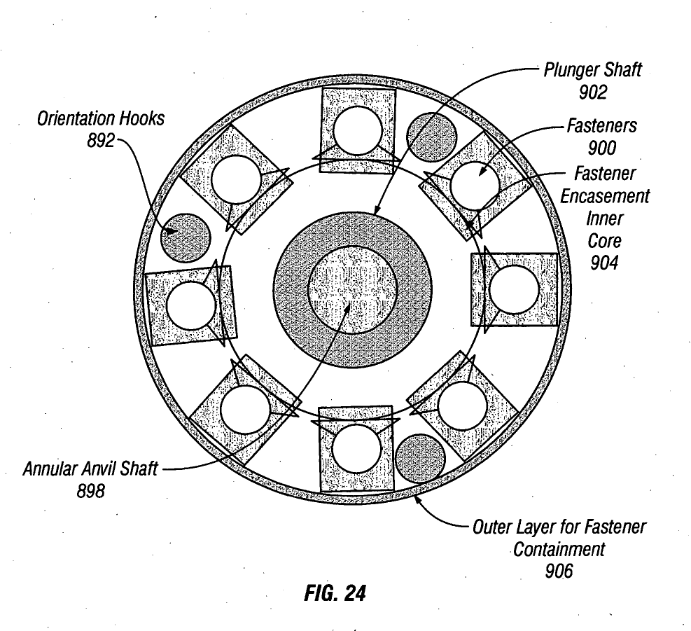

Referring now to FIG. 16, a close-up of the procedure of FIG. 15 is shown. As seen, the prosthetic valve annulus 814 is on one side of the aortic tissue T while balloon 802 is on an opposing side. The balloon 802 may be in a compressed stated so as to securely engage the tissue annulus T trapped therebetween. Arrows 810 indicate the direction in which the balloon 802 is being pulled. Sutures, fasteners, and/or clips may be advanced through the annulus as indicated by arrows 818.

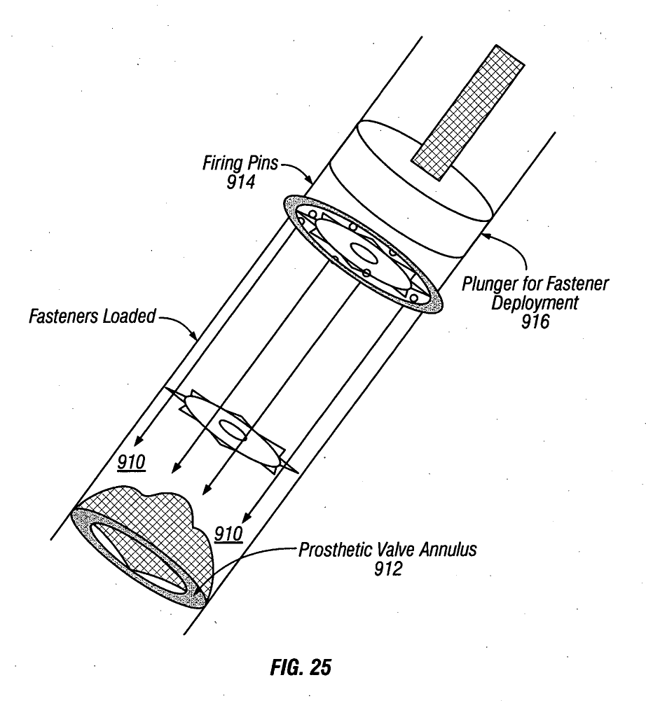

FIG. 17 shows one embodiment of the completed procedure. In this embodiment, a prosthetic valve annulus 814 is secured against annulus tissue T by clips 820. The rapid delivery and fastening of the prosthetic valve annulus 814 is enabled by apparatus 800 and the use of a balloon 802 or other anvil device to engage the annulus tissue T.

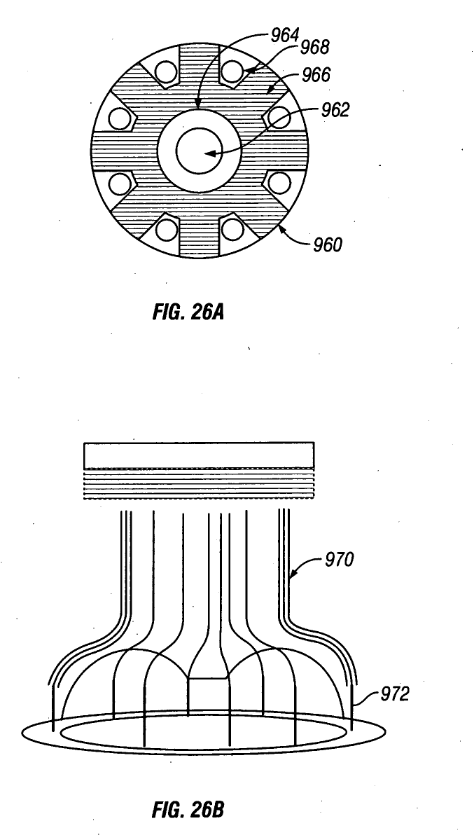

Referring now to FIGS. 18A-18B, it should be understood, that other devices may be used in place of balloon 802 to engage the tissue. As a nonlimiting example, a cone 821 as seen in FIG. 18A may be used to expand and engage the tissue. In a first configuration, the cone 821 may have a diameter of about 15mm while in a second configuration as seen in FIG. 18B, the cone may have a diameter of about 21-27 mm. It should be understood, these dimensions are purely illustrative and other dimensions may be used, depending on the size of the targeted valve or tissue.

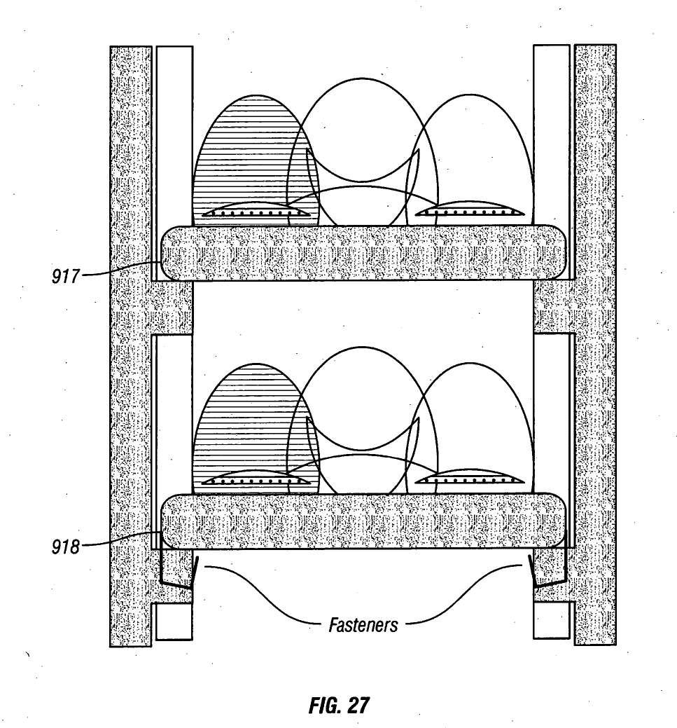

As another nonlimiting example, an expandable fan 820 as seen in FIG. 19 may also be used. A fan 820 may have a plurality of leaflets 822 which may be rotatably moved as indicated by arrows 824. The fan 820 will assume a substantially circular configuration as shown in phantom. In some embodiments, FIG. 20 shows that the leaflets 822 may be articulated between a first position where the leaflets 822 are aligned parallel to a longitudinal axis 830 of the apparatus 800 and a second position substantially perpendicular to the axis 830. It should be understood that the leaflets 822 may be moved to other angles other than being perpendicular to the axis 830. As a nonlimiting example, the shield may be shaped to guide clips or the leaflets of the device may be molded or shaped to guide the clips in a predetermined direction.

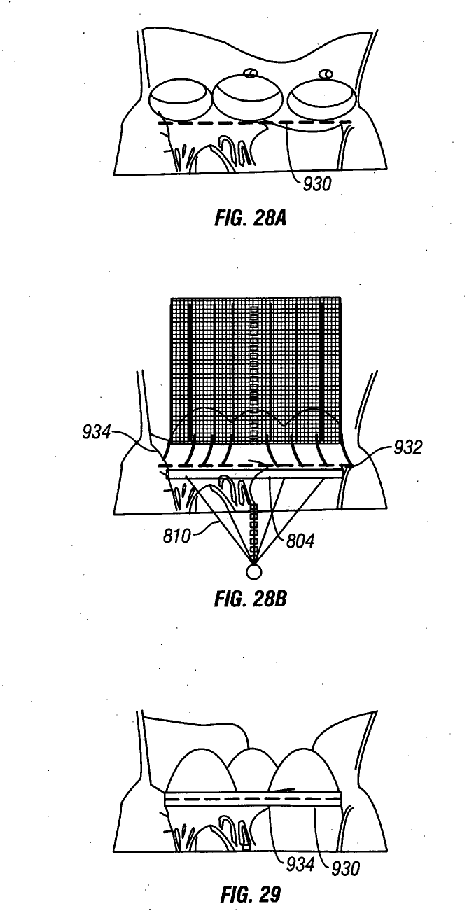

Referring now to FIG. 21, an embodiment of the present invention is shown for use with a stented bioprothesis or mechanical valve. The apparatus 860 includes plurality of orientation/apposition hooks 862 for positioning of the apparatus against the aortic annulus A. A prosthetic annulus 864 is mounted in the apparatus 860 and will be secured against the aortic annulus A. The prosthetic annulus 864 may be a part of a prosthetic valve 866. A valve protective housing 868 is optionally a part of apparatus 860 to protect the valve during delivery. When the apparatus 860 is properly positioned, the handle 870 may be advanced to move plunger 872 to deploy fasteners pre-loaded in the apparatus. In this particular embodiment, the fasteners are advanced in a substantially simultaneous manner.

Referring now to FIG. 22, yet another embodiment of the present invention is shown for use with a stented bioprothesis or mechanical valve. The apparatus 880 includes an aortic annular cone anvil 882 for use in positioning and/or engaging the aortic annulus A. The cone 882 may act as a support for trapping tissue or annulus A against a prosthetic annulus 864 on the apparatus 880. It should be understood that the tissue of annulus A could be an aortic annulus but is not limited as such and could be some other body tissue. A prosthetic valve 866 may be mounted on the apparatus 880 to provide a “one-shot” delivery of sutures through the valve 866 being attached to the tissue. A plurality of fasteners 869 may also be coupled to the apparatus 880. A prosthetic annulus 864 is mounted in the apparatus 880 and will be secured against the aortic annulus A. The prosthetic annulus 864 may be a part of a prosthetic valve 866. A valve protective housing 868 is optionally a part of apparatus 880 to protect the valve during delivery. An anvil tightening mechanism/handle 884 may be used to draw the anvil 882 to engage the tissue of the annulus A. A connector 886 is used to couple the anvil 882 to the handle 884. During use, the apparatus 880 may be positioned engage target tissue. A tightening device 884 may be retracted as indicated by arrow 885 or otherwise moved to draw the cone 821 to capture tissue between it and the annulus 840. When the apparatus 860 is properly positioned, the handle 870 may be advanced to move plunger 872 to deploy fasteners pre-loaded in the apparatus. In this particular embodiment, the fasteners are advanced in a substantially simultaneous manner.

Referring now to FIG. 23, a still further embodiment of the present invention is shown for use with a stentless bioprothesis. The apparatus 890 includes plurality of orientation hooks 892 for positioning of the apparatus against the aortic annulus A. A prosthetic annulus 894 is mounted in the apparatus 890 and will be secured against the aortic annulus A. The prosthetic annulus 894 may be a part of a prosthetic valve 896. When the apparatus 890 is properly positioned, the handle 870 may be advanced to move plunger 872 to deploy fasteners pre-loaded in the apparatus. In this particular embodiment, the fasteners are advanced in a substantially simultaneous manner. Some dimensions are shown in the figure for one embodiment of the apparatus 890.

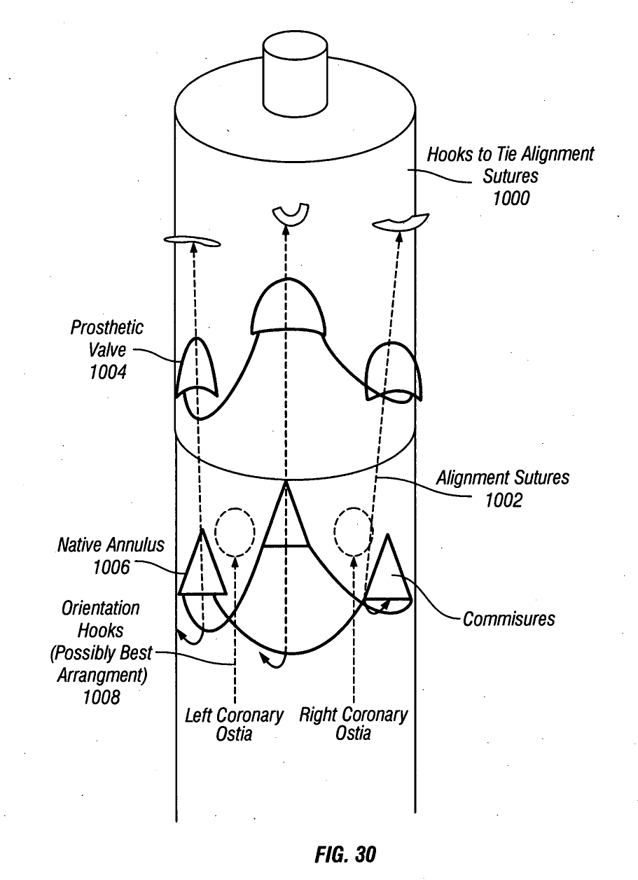

FIG. 24 shows a cross-section of the device of FIG. 23. The orientation hooks 892 are shown. The center of the apparatus 890 includes an annular anvil shaft 898 for drawing the anvil to engage tissue. A plurality of fasteners 900 are shown. A plunger shaft 902 is coupled to handle 890 and is used to advance the fasteners 900. A fastener encasement inner core 904 is shown along with an outer layer 906 for fastener containment. In some embodiments, the prosthetic annular design differ if they are stented or stentless and thus the arrange of the fasteners may also differ.

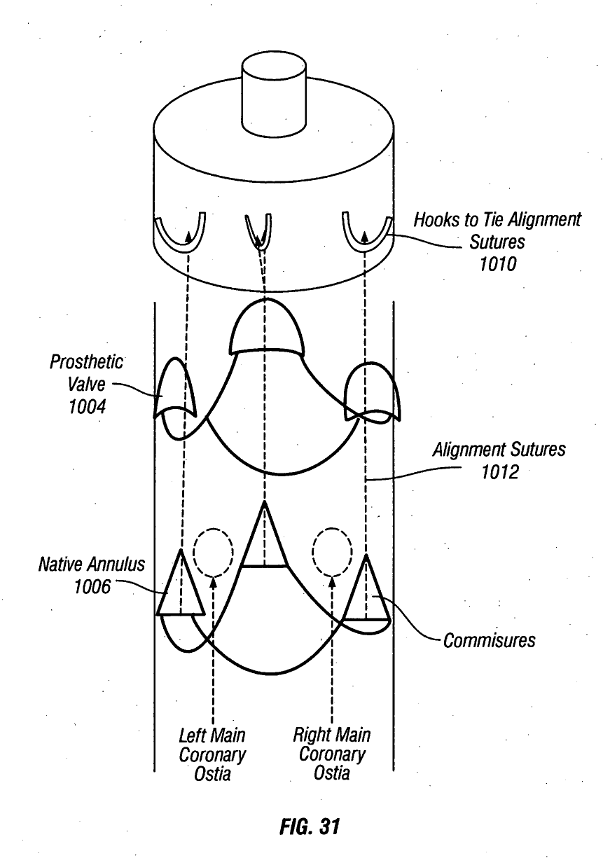

Referring now to FIG. 25, a cross-sectional view of the apparatus 909. As indicated by arrows 910, the fasteners loaded in the apparatus 909 may be advanced to engage the prosthetic valve annulus 912. A plurality of firing pins 914 may be mounted on a plunger 916 for engaging and advancing the fasteners.

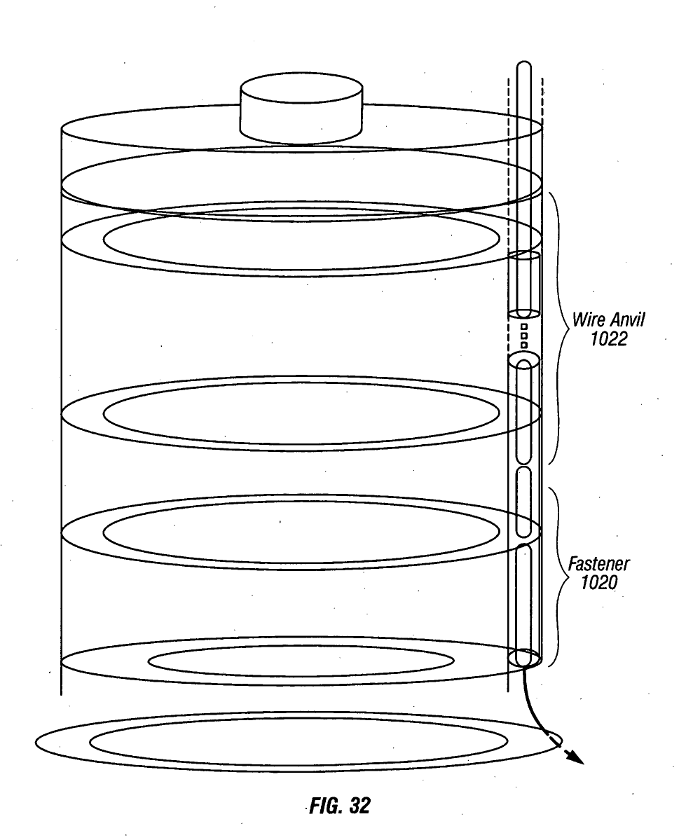

FIG. 26A shows a cross section of the apparatus 880. As seen in FIG. 26A, an outer sheath or outer layer 960 may be used for fastener containment. In this embodiment, the distal end of apparatus 880 is not free floating. This simplifies the delivery of the fasteners into the tissue. The apparatus 880 may be sized based on the targeted tissue, blood vessel, or valve. Shaft 962 may be used to guide the plunger shaft 964 to draw the cone 821 (as seen in FIG. 22) to engage the tissue. A fastener encasement inner core 966 may also be used to position fasteners 968 so that the fasteners do not need to be expanded to engage tissue.

FIG. 26B shows a vertical cross section of the apparatus 880. The sutures 970 attached to clips 972 is shown.

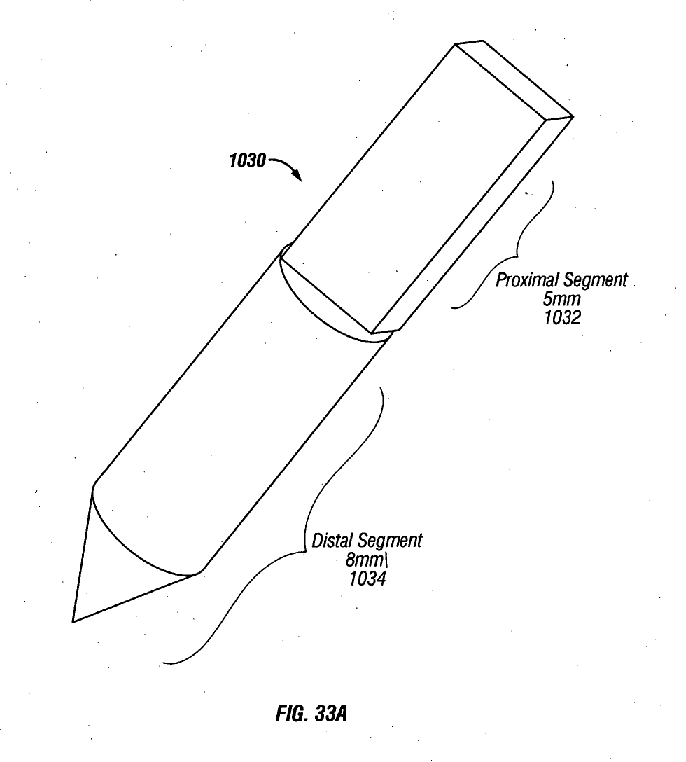

Referring now to FIG. 27, various placements of the prothetic annulus 917 and 918 are shown. In FIG. 27, annulus 917 is shown with a stented annular sewing ring. Annulus 918 is shown with a stentless annular sewing ring. The FIG. 27 shows the sewing rings 917 and 918 positioned above the ventriculo-arterial junction, in a supra-annulus position.

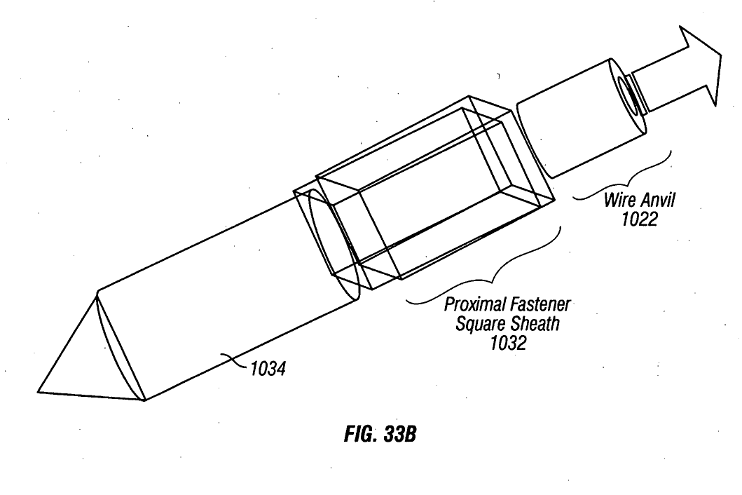

FIGS. 28A through 29 shows the anatomy around the intra-annular placement of a valve. FIG. 28A shows the VA junction 930 and one desired position for the valve device. Referring now to FIG. 28B, an apparatus 800 (only sutures 932 and cone 821 are shown) is positioned with a valve 934 to be positioned at the VA junction. The shield 804 may be used to guide the sutures 932 with their fasteners through the annulus of valve 934. Due to the relatively thin annulus, the apparatus 800 is desired since it can hold the annulus and penetrate through the annulus with a plurality of fasteners to simplify positioning and placement. FIG. 29 shows the valve 934 properly positioned at the VA junction 930.

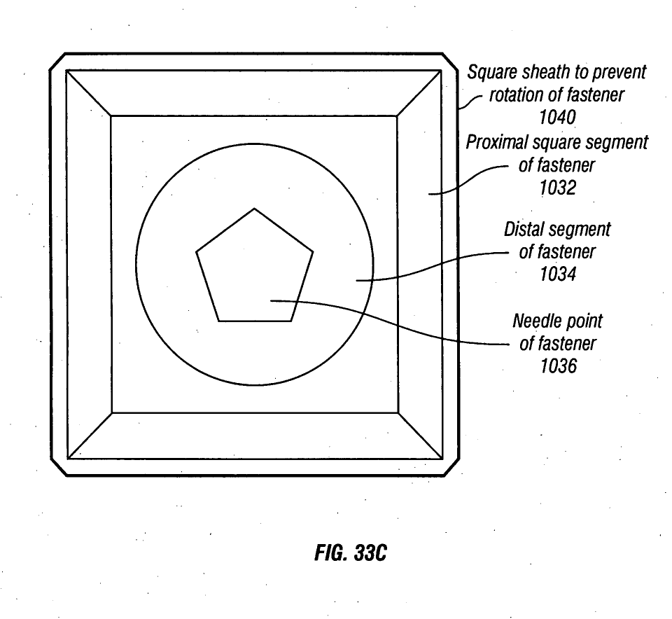

Referring now to FIG. 30, the alignment of sutures at the aortic valve base is shown. As seen, a plurality of hooks 1000 are provided for tying alignment sutures 1002. These sutures 1002 are used for aligning the prosthetic valve 1004 with the native annulus 1006, and as seen, the sutures 1002 are placed at the base of the aortic valve annulus. As seen, orientation hooks 1008 may be arranged to facilitate placement of sutures 1002.

FIG. 31 shows another method for placement of alignment sutures. As seen in FIG. 31, hooks 1010 are provided for the alignment sutures 1012 which may be placed through commissures C in the native annulus 1006. These sutures 1012 are used for aligning the prosthetic valve 1004 with the native annulus 1006.

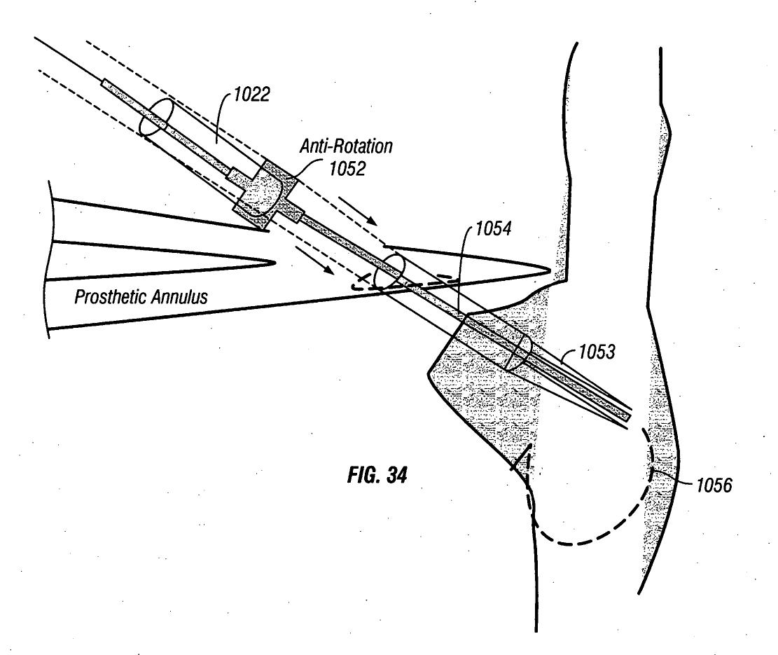

Referring now to FIG. 32, a diagram of a fastener driving mechanism is shown. The fastener 1020 may be driven forward by a wire anvil 1022 or drive pin. After the fastener 1020 exits the shaft, some embodiments of the fastener may assume a curved or other shape as appropriate.

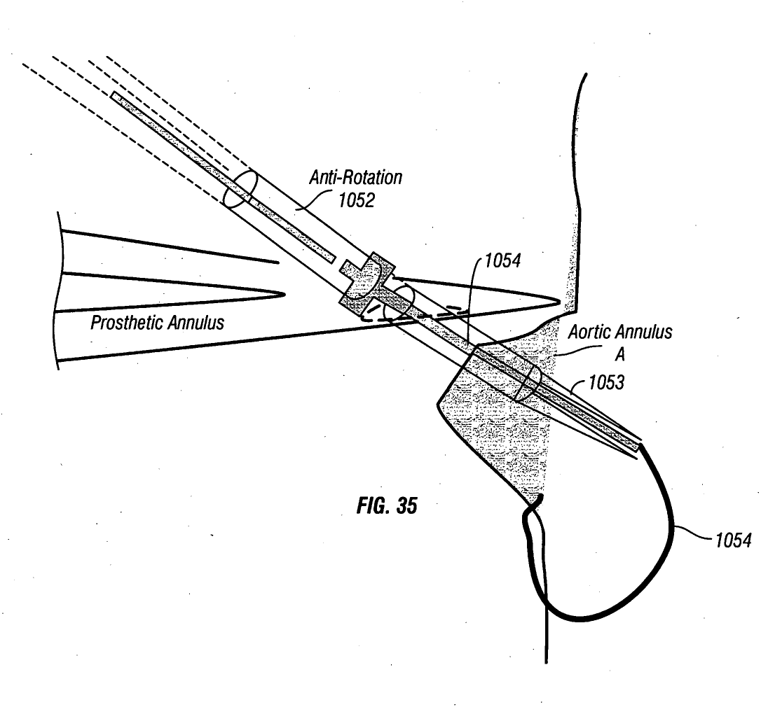

Referring now to FIGS. 33A-33C, one particular embodiment of a fastener 1030 is shown. As seen in FIG. 33A, the fastener 1030 may have a proximal segment 1032 that would have a rectangular cubed configuration to prevent rotation at the distal segment 1034 of the fastener. The distal segment 1034 would have a round configuration with a sharp distal end, similar to a surgical needle, to facilitate tissue penetration. It should be understood that the proximal section 1032 has a “key-ing” effect and allows the fastener to be properly oriented. This is advantageous since, in some embodiments, the fasteners 1030 are made of shape memory materials and the fasteners 1030 should be oriented to curve, bend, or assume their shape memory form in an orientation desired by the device. Without some method to control orientation, the fasteners 1030 may rotate or twist as they are being advanced through an apparatus 909 by wire anvil 1022, push rod, or other device as seen in FIG. 33B. By way of example and not limitation, a portion of the cross-section of the fastener may be square, polygonal, oval, triangular, rectangular, or other shape that prevent rotation about the longitudinal axis of the fastener during delivery.

FIG. 33C shows an axial, “head-on” view of the fastener 1030. The figure shows the sharpened, needle end 1036, a distal segment 1034, and the squared proximal segment 1032. A square sheath or channel 1040 is used to prevent rotation of the fastener 1030 as it is advanced. It should be understood, however, that a variety of different shapes such as but not limited to triangular, oval, hexagonal, polygonal, rectangular, trapezoidal, or the like may be used so long as the fasteners are properly oriented when then are delivered to the tissue site. In some embodiments, the wire anvil 1022 may contain a recess that is shaped to receive the shape of the proximal segment 1032 and thus also help in maintaining fastener orientation.

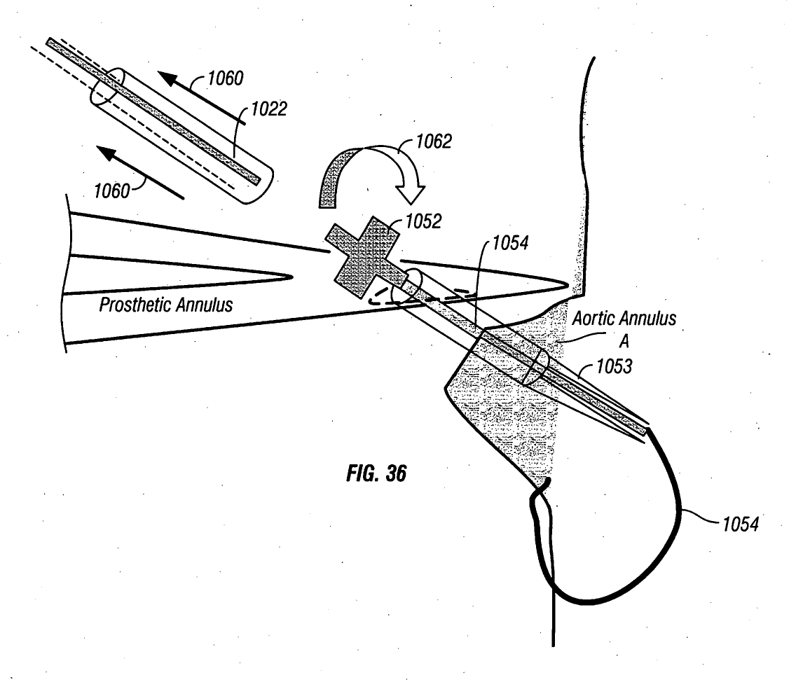

Referring now to FIG. 34, one method for the delivery of a fastener 1050 having a keyed proximal portion 1052 and a sharpened sheath portion 1053. As seen in FIG. 32, the fastener may exit the device at an outward facing orientation and penetrate a prosthetic annulus 894. In FIG. 34, the portion 1054 may be made of a shape memory material that will follow a path indicated by arrow 1056 shown in phantom. In this embodiment, the path is curved so as to secure the prosthetic annulus 894 to the tissue of the aortic annulus A.

FIG. 35 shows that as the portion 1054 is delivered outward, it assumes it shape-memory configuration and anchors into the tissue of the aortic annulus A. FIG. 36 shows the wire anvil or push rod 1022 being removed as indicated by arrows 1060. Proximal portion 1052 may also have a shape memory quality and may hook or bend as indicated by arrow 1062. FIG. 37 also shows that the proximal portion 1052 may be further advanced to embed in the sheath portion 1053.

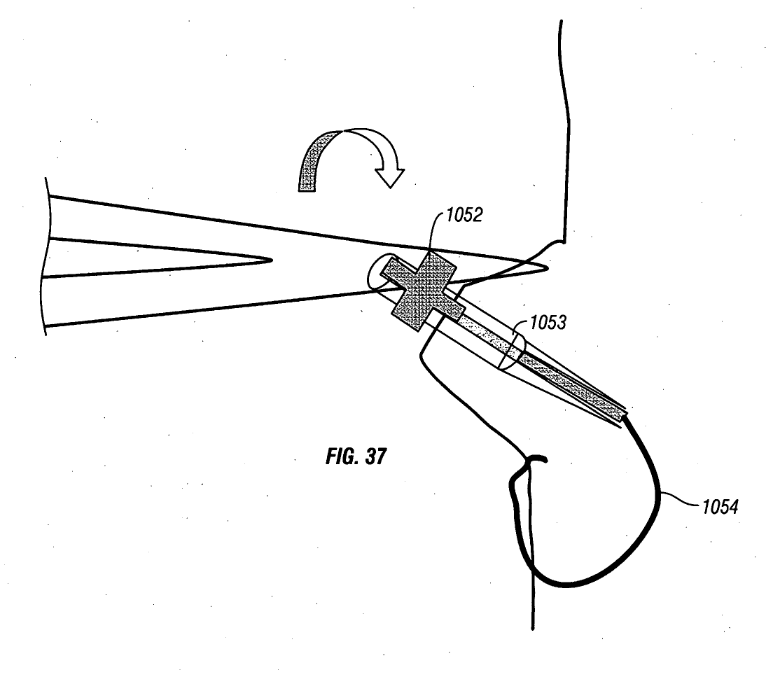

Referring now to a still further embodiment of the present invention, a resilient delivery device 1060 will now be described. FIG. 38 shows one embodiment of device 1060 where the device is spring-loaded so that it may be delivered through a tapered delivery conduit 1062 but resume its original shape after delivery as seen in FIG. 39. Fasteners 1064 may be positioned on the device 1060.

FIG. 40 shows that after the device 1060 is in position, fasteners 1064 may be advanced outward to engage the aortic annulus A, through downward motion of a plunger as indicated by arrow 1066. The fasteners 1064 move outward as indicated by arrow 1068.

FIG. 41 shows the fastener 1064 fully released from device 1060 and being retracted away as indicated by arrow 1070. The fastener 1064 can be used to secure a prosthetic annulus (not shown) at a position as indicated by line 1072.

FIG. 42 shows a cross-section of a stentless valve annulus. The circumference of a stentless annulus in a normal configuration is indicated by line 1074. The circumference of a stentless annulus in a deformed or compressed configuration is indicated by line 1076. A plurality of fasteners 1064 may be carried on or positioned with the annulus.

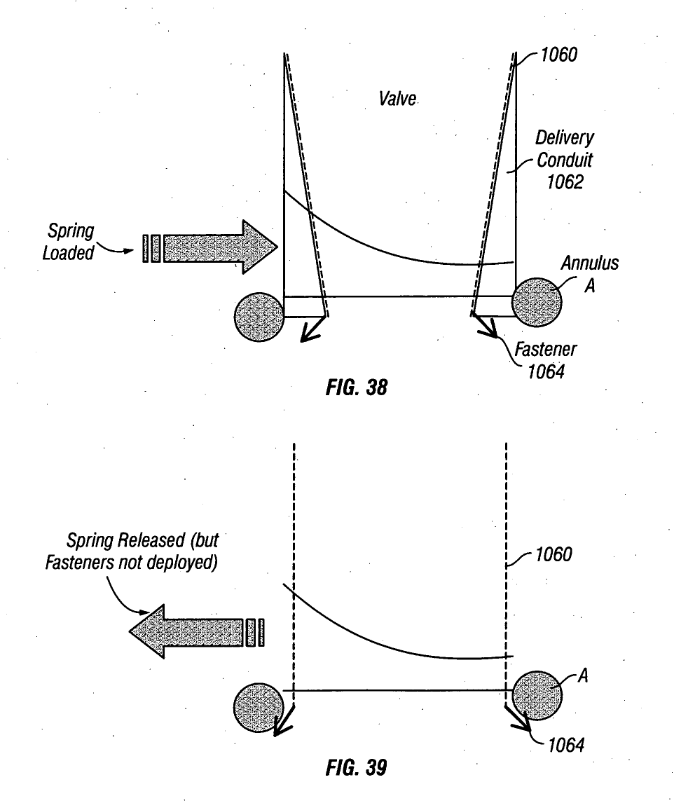

Referring now to FIGS. 43 and 44, yet another embodiment of the present invention will now be described. A prosthetic annulus 1100 is shown with a ring fastener unit 1102. It may be mounted within the ring of a stented valve. The ring fastener unit 1102 may have a plurality of penetrating members 1104. The penetrating members 1104 may be clips, needles, or other suitable device. The members 1104 may be deployed simultaneously, sequentially, or other sequence. The unit 1102 may facilitate delivery since the ring unit 1102 may be prepositioned relative to the prosthetic annulus 1100. Such a preloaded design may reduce the amount of time spent on the surgical procedure.

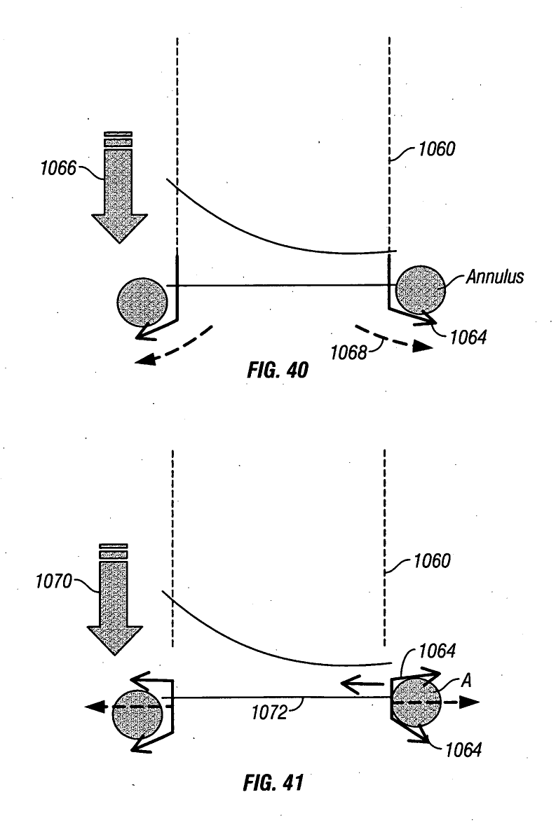

Referring now to FIGS. 45 and 46, another embodiment of the present invention will now be described. FIG. 45 is a cross-sectional view of one embodiment of a delivery device 1200 according to the present invention. The device 1200 includes a plunger 1202 having a plurality of pushing elements 1204. These pushing elements 1204 will pass through passageways 1206 in the fastener housing 1208 to push the fasteners in the passageways 1206 outward in the direction indicated by 1210. The fasteners will then pass through a sewing ring 1212 of the prosthetic valve 1214. The prosthetic valve 1214 may be pre-loaded and positioned inside the blood vessel 1220 having the target tissue area. For the device of FIG. 45, the valve prosthetic may be mounted along the inside surface of the fastener housing 1208. By way of example and not limitation, the fastener housing 1208 may have a circular, oval, polygonal, or other cross-sectional shape.

In one embodiment, the fastener housing 1208 may be advanced forward by a plunger or by user actuation to advance the sharpened guide tube 1211 to pierce the sewing ring 1212. After the tube 1211 pierces the sewing ring, the fastener may then be deployed. Some embodiments may actuate the fasteners without having the guide tubes 1211 penetrate the sewing ring. The use of a plunger will simultaneously eject a plurality of fasteners from the guide tubes 1211.

As seen in FIG. 45, the delivery device 1200 may be used with another embodiment of the tissue engagement device 1230 which is made to expand and engage the tissue at 1221. A cut-out section of aortic valve tissue 1220 is drawn to show its relationship to the position of the tissue engagement device 1230. In the present embodiment, the tissue engagement device 1230 may have a plurality of fingers 1232 that act as support elements. These fingers 1232 are coupled to a central disc 1234. FIG. 45 shows the tissue engagement device 1230 in an expanded configuration. A shaped plunger member 1240 is inserted into the center of the plurality of fingers 1232 and the shaped plunger member 1240 has a circumference sufficient to deflect the fingers 1232 to a position where the fingers are pushed radially outward as indicated by arrow 1242. By way of example and not limitation, the shaped plunger member 1240 may be rounded as shown in FIG. 45 or it may be, but is not limited to, shapes such as spheres, cones, wedges, cubes, polygons, or any single or multiple combination of the above. As seen in this embodiment, the tissue engagement device 1230 is expanded by drawing the fingers 1232 around the ball or pushing the ball into the tissue engagement device 1230. Although not limited to the following, the fingers 1232 may be made from nickel titanium alloy, stainless steel or polymer. In other embodiments, the tissue engagement device 1230 may have a hinge configuration with parts that may be articulated to expand.

Hinged fingers when in its undeployed position will remain at its minimum radial position to allow passage through the prosthetic valve opening once the tissue engagement device is passed through the valve or the aorta. The articulating hinged fingers can then be deployed to a larger radial configuration to support the tissue at point 1221. In some embodiments, the expandable device will contact the device to hold it in position. The device may include a support surface 1233 to contact the tissue. In some embodiments, the support surface 1233 may be used to align or stop the fastener housing.

In some embodiments, the fingers 1232 may be coupled together by a mesh material such a DARON™, Dacron™, a firm rubber substance, GORTEX™, any combination of the above, or similar substances to capture debris that may be created by the valve repair procedure. In some embodiments, the fasteners will align to extend outward in the gaps between fingers 1232 so that the fingers do not interfere with deployment of the fasteners.

FIG. 46 shows an exploded perspective view of the embodiment of FIG. 45. The FIG. 46 also shows that a handle 1250 may be included to facilitate the pushing of plunger 1202 to eject the fasteners and attach the prosthesis 1214 to target tissue. FIG. 46 shows the prosthetic valve 1214 on the inside of the fastener housing 1208. In this embodiment of the delivery device 1200, the fasteners will embed through the shoulder or sewing ring 1212 of the valve 1214.

As seen in FIG. 46, the needles may pass through a straight portion when it exits. In such a configuration, it may be desirable to key the passageway and the cross-section of the fastener so that the fasteners will extend outward and curve in the desired direction. The present embodiment passes through the top of the shoulders or sewing rings and then hooks.

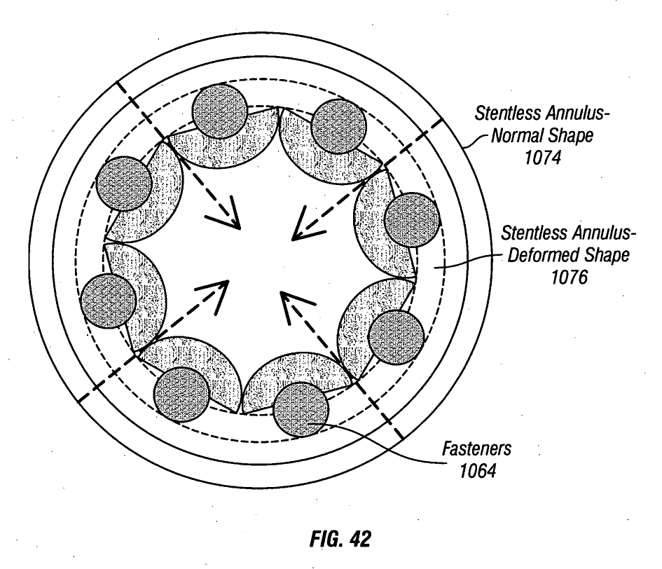

Referring now to FIG. 47, one embodiment of the tissue engagement device 1230 is shown. In this embodiment, the shaped plunger member 1240 may be coupled to a shaft 1260. The shaft 1260 may be fixed along the longitudinal axis of the device 1200. In other embodiments, the shaft 1260 may be slidably mounted within the device 1200. The shaft 1260 may be slidably mounted over another shaft 1262 which is coupled to the tissue engagement device 1230. This allows the device 1230 to traverse. The shaped plunger member 1240 and the device 1230 may both translate or move relative to each other. This telescoping configuration allows the ball-shaped plunger member 1240 to be moved inside the tissue engagement device 1230 to expand the fingers 1232 outward. Other embodiments may have the shaft 1260 coupled to the device 1230 and the shaped plunger member 1240 coupled to shaft 1262.

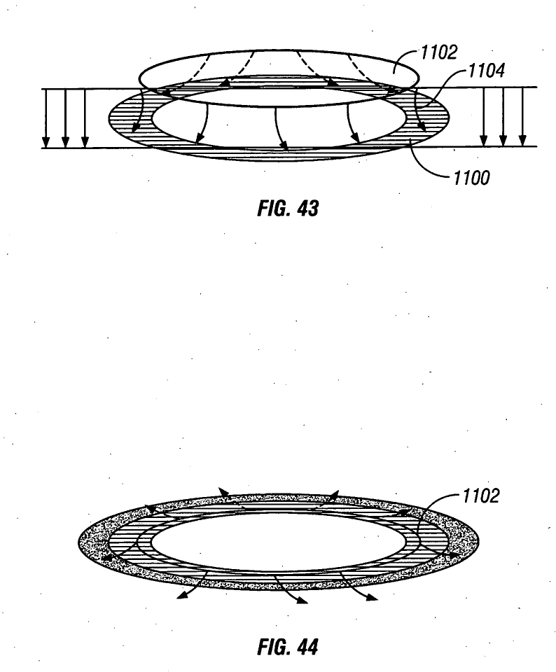

Referring now to FIG. 48, the plunger 1202 is shown with the fastener pushers 1204 engaging the fastener housing 1208. The fasteners are held inside the housing 1208 prior to being deployed for use. In one embodiment, the fasteners are made of pre-shaped superelastic nitinol material which is held in place within the fastener housing due to friction force exerted by the pre-shaped material.

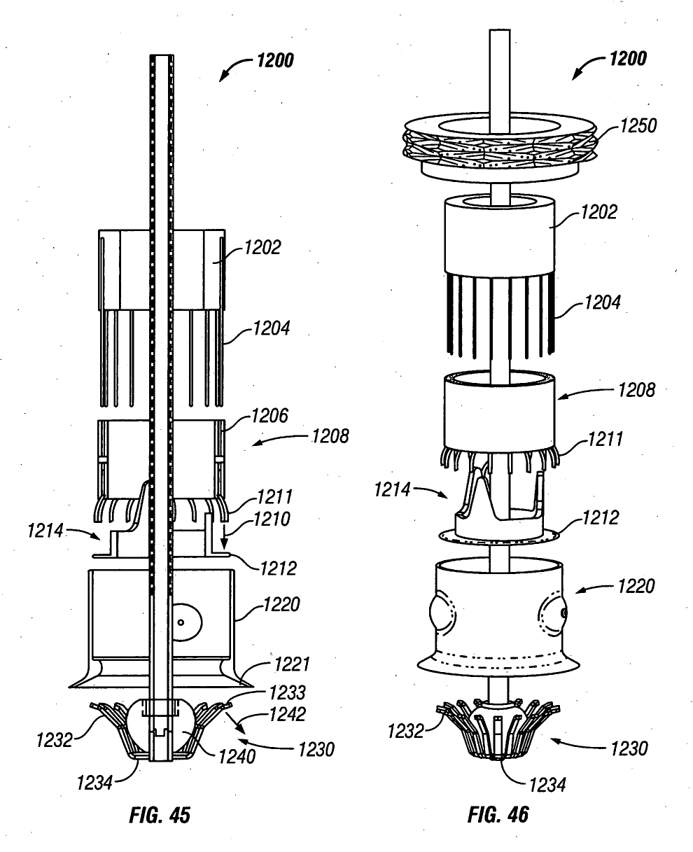

Referring now to FIGS. 49 and 50, perspective view of the device 1200 are shown. FIG. 49 shows the device 1200 fully assembled and in a configuration where the plunger 1200 has been advanced towards a distal end of the device 1200 to deploy the fasteners. As seen in FIG. 49, the handle 1250 may be used to push on pins 1270 to advance the plunger 1202. The pins 1270 may travel down a straight groove 1272 formed on an outer housing 1274. FIG. 49 also shows that for the present embodiment, the tissue engagement device 1230 may be sized to be deliverable into the blood vessel 1220.

FIG. 50 shows an exploded perspective view where the pins 1270 are shown to engage the plunger 1202 via holes 1276 formed in the plunger. In this view, the prosthetic valve is inside the cut-out aortic section, which is supported from the bottom with the tissue engagement device 1230 at location 1221 when the fasteners are deployed to engage the prosthetic valve into the aortic tissue 1220.

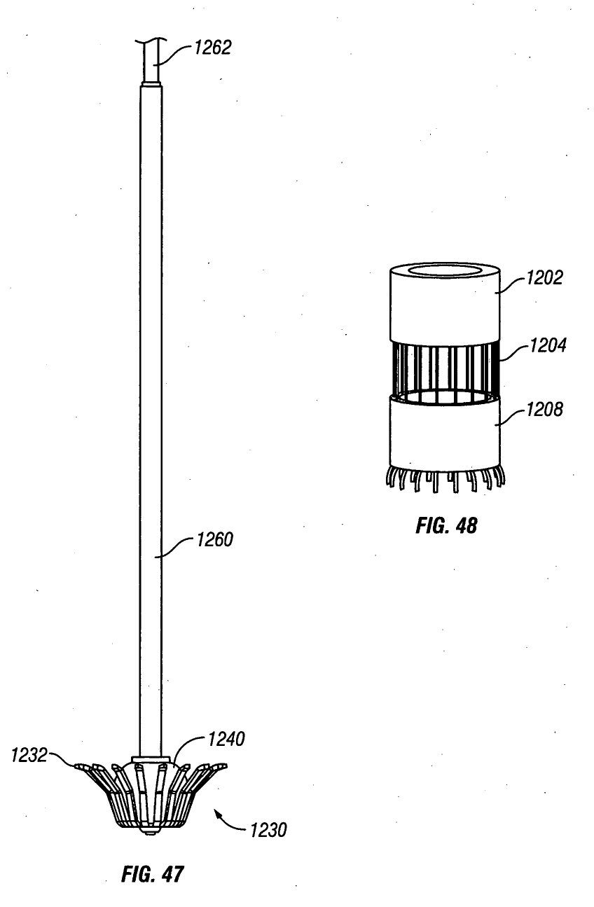

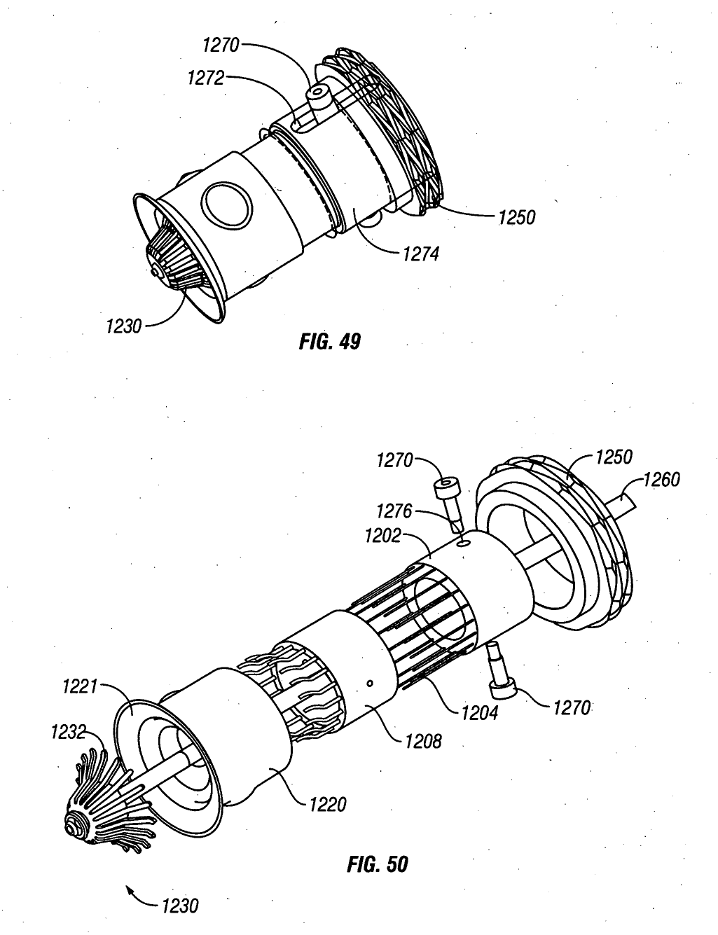

Referring now to FIG. 51, yet another embodiment of the present invention will now be described. FIG. 51 shows a cross-section view of a prosthetic delivery device 1300. The device 1300 may have a fastener housing 1308 with passageways 1306 for guiding the fastener 1310 in a desired direction. In this particular embodiment, the valve 1314 is mounted about the fastener housing 1308. As will be described in more detail in FIG. 52, the fasteners 1310 will pass through the valve and then into the target tissue.

This embodiment uses a support device 1330 having a plurality of hinged fingers 1332 attached at a hinge point 1334 to a base 1336. A slider 1338 is moveable relative to base 1336 and is slidably mounted over the shaft 1340. The slider 1338 may be moved to engage an edge 1342 of the finger 1332 to urge the finger to a position that expands the device 1330. The fingers 1332 may be biased to retract as indicated by arrow 1334 to its original position to configure the device 1330 in a collapsed configuration. The fingers have may have a support surface near the distal end of each finger to facilitate contact with tissue and/or the prosthesis.

FIG. 52 shows a perspective view of a valve 1314 that does not include a sewing ring. The valve 1314 will be slidably mounted about the housing 1308.

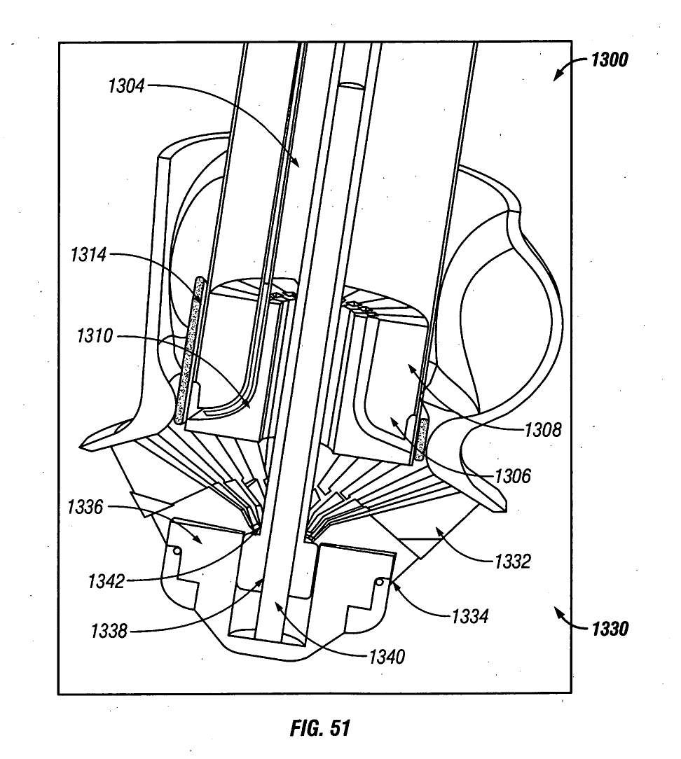

FIG. 53 shows an enlarged cross-section view of the embodiment of device 1330 from FIG. 51. The fastener 1310 and push rod 1304 are more clearly shown. As seen in FIG. 53, the fastener 1310 and push rod 1304 are actually housed inside a hollow piercing member 1340. The hollow piercing member 1340 may act as a guide tube and have a portion near the sharpened tip that is configured to be easily bendable. By way of example and not limitation, portions can be removed from the member 1340 to facilitate bending. The hollow piercing member 1340 may also be made from two pieces, which may then be integrated together. This allows for a more expensive sharpened tip portion coupled to a less expensive tube portion which can extend proximally to a plunger or other driver for actuation. There can be a mechanical stop to limit the travel of the plunger which actuates the member 1340. In some embodiments, a travel or 3-4 mm is sufficient for piercing through the valve prosthesis and into the tissue.

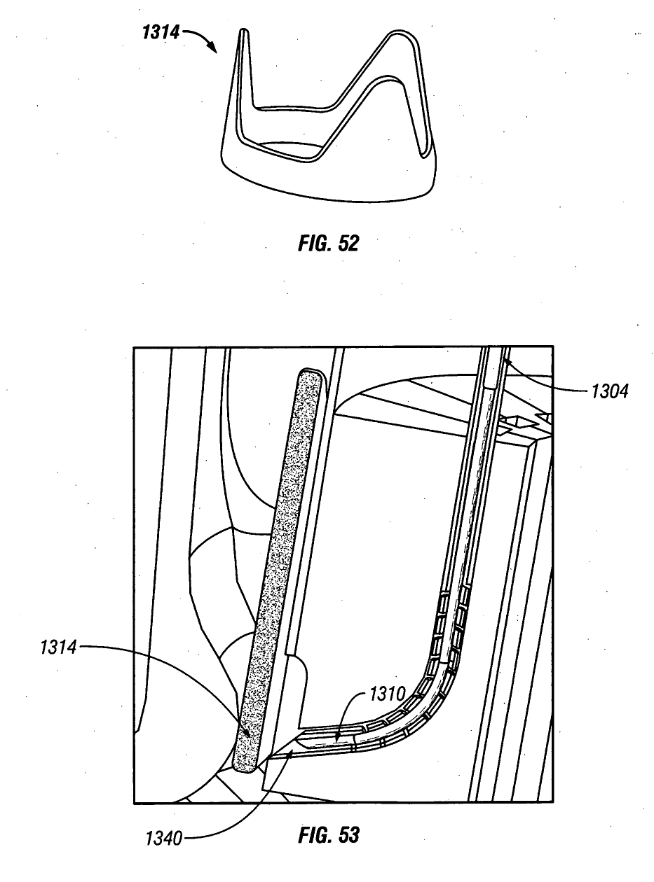

As seen more clearly in FIG. 54, the hollow piercing member 1340 may be configured to curve within the passageway 1306 by having a plurality of cut-outs 1342 along the portion of the hollow piercing member 1340 that will curve with the passageway.

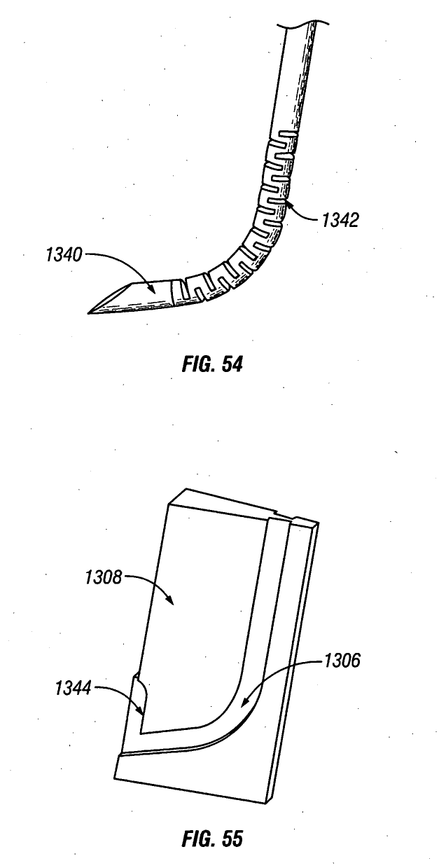

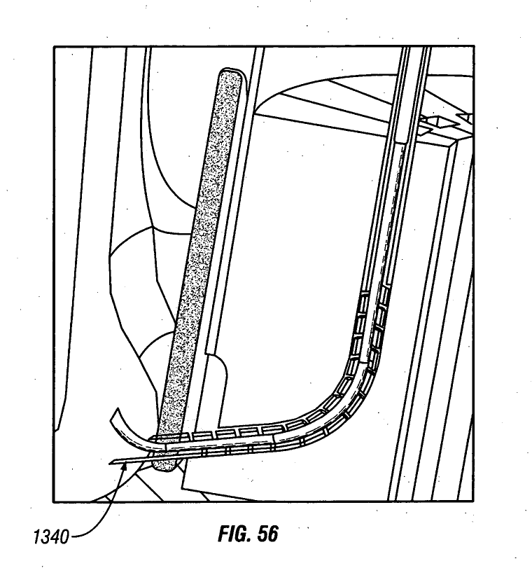

FIG. 55 shows how passageway 1306 is curved to guide the hollow piercing member 1340 and the fastener 1310. The fastener housing 1308 may include a cavity area near the exit of the passageway 1306. As will be seen more clearly in FIG. 56, this provides clearance for the fastener to pass through the valve material at one location and loop back through the valve at a second location.

Referring now to FIG. 56, one method of deploying a fastener 1310 will now be described. As seen in FIG. 56, the hollow piercing member 1340 is extended outward from the passageway 1306. By way of example and not limitation, the member 1340 may extend a distance of about 3 mm. In the present method, the member 1340 will piercing through the valve 1314 and into the target tissue. Once the member 1340 has reached a desired penetration depth, the fastener 1310 is then deployed. The hollow guide member 1340 guides the member through the valve 1314 and prevents fastener 1310 from curving too early. This allows the fastener 1310 to penetrate more deeply into the target tissue and provide a more secure anchor. As seen in FIG. 56, the fastener 1310 is beginning to curve and point back towards the valve 1314.

Referring now to FIG. 57, the fastener 1310 is shown in a curved configuration. The fastener 1310 is shown to have formed two loops, passing through the valve material four times. The cavity 1344 allows for the loops to be formed without interference from the housing 1308. The FIG. 57 also shows the fastener piercing the valve at two different locations as it loops through the valve prosthetic. Some embodiments may pierce at more than two different locations, depending on how many loops are formed and where the fastener reenters the valve prosthetic.

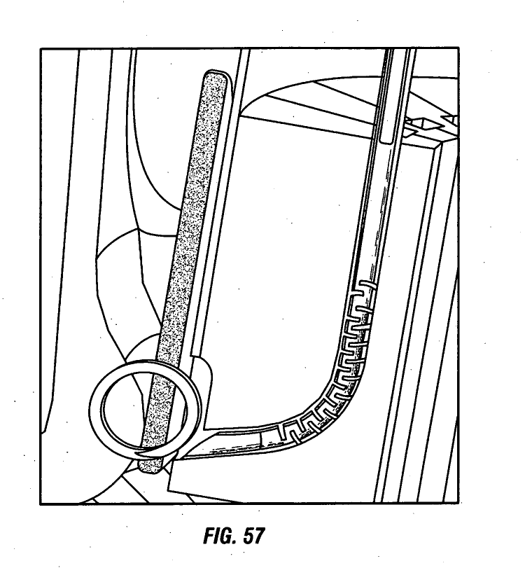

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. The number of fasteners on the delivery may include but are not limited to at least 5, 6, 7, 8, 9, 10, 11, 12, 13, 14, 15, 16, 17, 18, 19, 20, or more fasteners. Some fasteners may have sharpened tips while others may be blunt or there may be combinations of both. With any of the above embodiments, the fasteners may each form 1, 2, or more loops to secure the prosthesis to the tissue. Some alterative may use a support device that is not expandable but may be anchored by some other method such as via hooks with extend outward or other anchor to secure the support device in place. Still others may simply be a device large enough to pass through the annular opening, but not expand any further. The user holds the device in place to guide the delivery device in position. With any of the embodiments above, some may have a plunger 1400 that actuates one subset of push rods, while another plunger 1402 actuates another subset as seen in FIG. 58. Still other embodiments may use a cam device 1404 to sequentially actuate each pushrod as seen in FIG. 59. A twisting action as indicated by arrows 1406 may be used by the user to eject the fasteners.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

1. A device for use in attaching a valve prosthesis to a target tissue, the device comprising:
a fastener housing;
a plurality of fasteners ejectably mounted in the fastener housing;
wherein the valve prosthesis is releasably mounted to a distal portion of the fastener housing; and
a tissue engagement device movable along a longitudinal axis of the fastener housing and having a surface to engage tissue disposed between the tissue engagement device and the valve prosthesis,
wherein the tissue engagement device is movable from a first position to a second position to engage tissue and
wherein the tissue engagement device is expandable from a first configuration to a second configuration.

2. The device of claim 1 further comprising a plunger and a plurality of fastener pushers coupled to the plunger;
wherein the plunger is movable along a longitudinal axis of the device;
wherein the fastener housing includes a plurality of passageways for receiving the fastener pushers and for guiding the fastener pushers to eject the fasteners when the plunger is moved towards a distal end of the housing.

3. The device of claim 1 wherein the tissue engagement device is expandable from a compressed configuration to an expanded configuration.

4. The device of claim 1 wherein the tissue engagement device is radially expandable from a compressed configuration to an expanded configuration.

5. The device of claim 1 wherein the tissue engagement device is formed from a plurality of elongate support elements extending radially outward from a central disc, said support elements movable from a first position to a second, expanded position.

6. The device of claim 1 wherein the tissue engagement device is configured to be engaged by a shaped plunger member have a circumference sized to move support elements on the tissue engagement device from a first position to second, expanded position.

7. The device of claim 7 wherein the shaped plunger member is sphere-shaped having a diameter sufficient to move said support element to the second position.

8. The device of claim 7 wherein the shaped plunger member is mounted to shaft that is slidably mounted within a shaft coupled to the tissue connection device, said shape member movable relative to the tissue connection device.

9. The device of claim 7 wherein the shaped plunger member is mounted to shaft that is slidably mounted over a shaft coupled to the tissue connection device, said shape member movable relative to the tissue connection device.

10. The device of claim 1 wherein the tissue engagement device is inflatable.

11. The device of claim 1 wherein prosthesis includes a sewing ring.

12. The device of claim 1 wherein prosthesis includes a sewing ring positioned around an outer circumference of the prosthesis.

13. The device of claim 1 wherein passageways in the fastener housing are configured to direct the fasteners outward through a sewing ring on the prosthesis and then into the target tissue.

14. The device of claim 1 further comprising a shaft extending through the fastener housing and coupled to the tissue engagement device.

15. The device of claim 1 further comprising a hollow, elongate member having a sharpened tip, wherein the elongate member is slidably mounted to move outward and through the tissue.

16. The device of claim 1 wherein the fasteners are made of a shape memory material.

17. The device of claim 1 wherein the fasteners assumes a coiled configuration when released from passageways in the fastener housing.

18. The device of claim 1 wherein the fastener housing has a fixed outer diameter.

19. The device of claim 1 wherein passageways defined by the fastener housing do not move relative to another passageway in the fastener housing.

20. The device of claim 1 wherein the tissue engagement device in a collapsed state is sized to pass through an opening of an annulus created by removing valve leaflets.

21. The device of claim 1 wherein the tissue engagement device in an expanded state has a maximum diameter no more than about 3 mm greater than a maximum diameter of the valve prosthesis.

22. The device of claim 1 wherein the tissue engagement device in an expanded state has a maximum diameter no more than about 12% greater than a maximum diameter of the valve prosthesis.

23. The device of claim 1 wherein the valve prosthesis is mounted along an inner surface of the fastener housing prior to attachment to target tissue.

24. The device of claim 1 wherein the valve prosthesis is mounted along an inner surface of the fastener housing with the sewing ring of the prosthesis positioned to extend beyond the outer surface of the fastener housing prior to attachment to target tissue.

25. A valve delivery device for use with a stentless valve prosthesis comprising:
a fastener housing;
a plurality of fasteners ejectably mounted in the fastener housing, wherein said fasteners when ejected will couple the prosthesis to target tissue; and
wherein the valve prosthesis is releasably mounted about the fastener housing;
an support device movable along a longitudinal axis of the fastener housing to engage tissue and to align the valve prosthesis, wherein the engagement device is expandable from a first configuration to a second, expanded configuration to facilitate engagement against the tissue.

26. The device of claim 25 wherein passageways defined by the fastener housing are each shaped to direct the fasteners to extend radially outward.

27. The device of claim 25 wherein passageways defined by the fastener housing are each curved to direct the fasteners to exit the passageway in a direction away from the longitudinal axis of the device.

28. The device of claim 25 wherein exits of each passageway in the fastener housing direct each of the fasteners to extend through an inner surface of the prosthesis prior to engaging the target tissue.

29. The device of claim 25 wherein exits of each passageway of the fastener housing includes a cavity or cut-out at the passageway configured to allow the fastener to exit from the passageway, penetrate the valve prosthesis at a first location, penetrate tissue, pass back through the valve at a second location, pass into the cavity, pierce back into valve material and into the tissue.

30. The device of claim 25 wherein valve prosthesis is without a sewing ring.

31. The device of claim 25 further comprising a hollow piercing member configured to be slidably mounted within the passageway defined by the fastener housing.

32. The device of claim 25 further comprising a hollow piercing member with a sharpened tip and slidably mounted within the passageway defined by the fastener housing.

33. The device of claim 25 further comprising a hollow piercing member with a sharpened tip and slidably mounted within the passageway defined by the fastener housing, wherein the fastener is slidably mounted within the hollow piercing member.

34. The device of claim 25 further comprising a hollow piercing member with a sharpened tip and slidably mounted within the passageway, wherein the piercing member comprises an elongate tube with a bendable portion near the sharpened tip.

35. The device of claim 25 wherein the prosthetic valve is an aortic stentless valve.

36. A method for placing a valve prosthesis to engage a target tissue comprising:
a fastener housing;
a plurality of fasteners ejectably mounted in the fastener housing, wherein said fasteners when ejected will couple the prosthesis to target tissue; and
means for tissue engagement wherein said means are movable along a longitudinal axis of the fastener housing to engage tissue disposed between the tissue engagement device and the valve prosthesis, wherein the engagement device is expandable from a first configuration to a second, expanded configuration to facilitate engagement against the tissue.

37. A method for placing a valve prosthesis to engage a target tissue comprising:
providing a valve prosthesis delivery device comprising a plurality of fasteners, a fastener housing, and an expandable tissue support device;
moving the tissue support device in a collapsed state through an annulus of target tissue;
expanding the tissue support device from the collapsed state to an expanded state;
pulling the tissue support device to engage a bottom surface of the target tissue;
stabilizing the annulus in preparation for delivery of the prosthetic device which includes the plurality of fasteners;
sliding the fastener housing towards the target tissue, said fastener housing incorporating the prosthetic valve on the distal surface of the annulus;
piercing the prosthetic valve with a shaped fastener guide;
pushing a plunger towards a distal end of the delivery device, said plunger having a plurality of push rods to eject a plurality of fasteners outward along a path to attach the valve to the target tissue.

38. The method of claim 37 further comprising compressing tissue between the fastener housing and the prosthetic valve when the fastener housing is engaged with the target tissue.

39. The method of claim 37 further comprising removing the fastener housing from the target tissue while leaving said prosthetic valve behind and attached to the target tissue.

40. The method of claim 37 wherein the target tissue is the annulus of an aortic valve with the valve leaflets removed.

41. The method of claim 37 further comprising piercing the target tissue without expanding the outer circumference of the fastener housing.

42. A kit comprising:
a valve prosthesis delivery device having a tissue engagement device;
a valve prosthesis;
instructions for use setting forth the method of claim 37;
a container sized to house the valve prosthesis delivery device, the valve prosthesis, and the instructions for use.

43. A method of securing a prosthesis to a target tissue, the method comprising:
delivering a support device through an opening defined by a valve annulus, said support device having a shaft coupled to the device;
expanding the support device from a collapsed configuration to an expanded configuration wherein the support device in the expanded configuration allows a support surface to be positioned at a circumference sufficient to support tissue;
positioning a prosthesis delivery device by guiding the device along the shaft of the support device;
ejecting a plurality of fasteners to secure the prosthesis to a target tissue; and
removing the support device and the delivery device while leaving the prosthesis attached to the target tissue.

44. The method of claim 43 wherein the prosthesis delivery device is pushed along the shaft until the delivery device contacts the support device.

45. The method of claim 43 wherein the prosthesis delivery device is pushed along the shaft until tissue is gripped between the delivery device and the support device.

46. A device comprising:
a housing;
a plurality of fasteners ejectable from the housing; and
a support device movable from a first position to a second position to facilitate delivery of said fasteners or of a prosthetic; and
wherein the support device is expandable from a first configuration to a second configuration.

47. A device comprising:
a ring;
a plurality of fasteners coupled to the ring; and
a support device movable from a first position to a second position to facilitate delivery of said fasteners to attach the prosthetic to target tissue; and
wherein the support device is expandable from a first configuration to a second configuration;
wherein the fasteners on the ring are each used to pierce tissue to couple the ring to the tissue.

48. A kit for use with a valve prosthesis, the kit comprising:
a valve prosthesis delivery device having a support device;
instructions for use setting forth the method of claim 43;
a container sized to house the valve prosthesis delivery device, the valve prosthesis, and the instructions for use.
